SEQUENCE LISTING (24 sequences)

SEQ ID NO: 1 - DNA, 1407 bp, Unknown
  Description of Unknown Acetaldehyde dehydrogenase polynucleotide
atgaataaagacacactaatacctacaactaaagatttaaaagtaaaaacaaatggtgaaaacattaatttaaagaactacaaggataattcttcatgtttcggagtattcgaaaatgttgaaaatgctataagcagcgctgtacacgcacaaaagatattatcccttcattatacaaaagagcaaagagaaaaaatcataactgagataagaaaggccgcattacaaaataaagaggtcttggctacaatgattctagaagaaacacatatgggaagatatgaggataaaatattaaaacatgaattggtagctaaatatactcctggtacagaagatttaactactactgcttggtcaggtgataatggtcttacagttgtagaaatgtctccatatggtgttataggtgcaataactccttctacgaatccaactgaaactgtaatatgtaatagcataggcatgatagctgctggaaatgctgtagtatttaacggacacccatgcgctaaaaaatgtgttgcctttgctgttgaaatgataaataaggcaattatttcatgtggcggtcctgaaaatctagtaacaactataaaaaatccaactatggagtctctagatgcaattattaagcatccttcaataaaacttctttgcggaactgggggtccaggaatggtaaaaaccctcttaaattctggtaagaaagctataggtgctggtgctggaaatccaccagttattgtagatgatactgctgatatagaaaaggctggtaggagcatcattgaaggctgttcttttgataataatttaccttgtattgcagaaaaagaagtatttgtttttgagaatgttgcagatgatttaatatctaacatgctaaaaaataatgctgtaattataaatgaagatcaagtatcaaaattaatagatttagtattacaaaaaaataatgaaactcaagaatactttataaacaaaaaatgggtaggaaaagatgcaaaattattcttagatgaaatagatgttgagtctccttcaaatgttaaatgcataatctgcgaagtaaatgcaaatcatccatttgttatgacagaactcatgatgccaatattgccaattgtaagagttaaagatatagatgaagctattaaatatgcaaagatagcagaacaaaatagaaaacatagtgcctatatttattctaaaaatatagacaacctaaatagatttgaaagagaaatagatactactatttttgtaaagaatgctaaatcttttgctggtgttggttatgaagcagaaggatttacaactttcactattgctggatctactggtgagggaataacctctgcaaggaattttacaagacaaagaagatgtgtacttgccggctaa

SEQ ID NO: 2 - protein, 468 aa, Unknown
  Description of Unknown Acetaldehyde dehydrogenase polypeptide
MNKDTLIPTTKDLKVKTNGENINLKNYKDNSSCFGVFENVENAISSAVHAQKILSLHYTKEQREKIITEIRKAALQNKEVLATMILEETHMGRYEDKILKHELVAKYTPGTEDLTTTAWSGDNGLTVVEMSPYGVIGAITPSTNPTETVICNSIGMIAAGNAVVFNGHPCAKKCVAFAVEMINKAIISCGGPENLVTTIKNPTMESLDAIIKHPSIKLLCGTGGPGMVKTLLNSGKKAIGAGAGNPPVIVDDTADIEKAGRSIIEGCSFDNNLPCIAEKEVFVFENVADDLISNMLKNNAVIINEDQVSKLIDLVLQKNNETQEYFINKKWVGKDAKLFLDEIDVESPSNVKCIICEVNANHPFVMTELMMPILPIVRVKDIDEAIKYAKIAEQNRKHSAYIYSKNIDNLNRFEREIDTTIFVKNAKSFAGVGYEAEGFTTFTIAGSTGEGITSARNFTRQRRCVLAG

SEQ ID NO: 3 - DNA, 1173 bp, Unknown
  Description of Unknown Alcohol dehydrogenase polynucleotide
gtggttgatttcgaatattcaataccaactagaatttttttcggtaaagataagataaatgtacttggaagagagcttaaaaaatatggttctaaagtgcttatagtttatggtggaggaagtataaagagaaatggaatatatgataaagctgtaagtatacttgaaaaaaacagtattaaattttatgaacttgcaggagtagagccaaatccaagagtaactacagttgaaaaaggagttaaaatatgtagagaaaatggagttgaagtagtactagctataggtggaggaagtgcaatagattgcgcaaaggttatagcagcagcatgtgaatatgatggaaatccatgggatattgtgttagatggctcaaaaataaaaagggtgcttcctatagctagtatattaaccattgctgcaacaggatcagaaatggatacgtgggcagtaataaataatatggatacaaacgaaaaactaattgcggcacatccagatatggctcctaagttttctatattagatccaacgtatacgtataccgtacctaccaatcaaacagcagcaggaacagctgatattatgagtcatatatttgaggtgtattttagtaatacaaaaacagcatatttgcaggatagaatggcagaagcgttattaagaacttgtattaaatatggaggaatagctcttgagaagccggatgattatgaggcaagagccaatctaatgtgggcttcaagtcttgcgataaatggacttttaacatatggtaaagacactaattggagtgtacacttaatggaacatgaattaagtgcttattacgacataacacacggcgtagggcttgcaattttaacacctaattggatggagtatattttaaataatgatacagtgtacaagtttgttgaatatggtgtaaatgtttggggaatagacaaagaaaaaaatcactatgacatagcacatcaagcaatacaaaaaacaagagattactttgtaaatgtactaggtttaccatctagactgagagatgttggaattgaagaagaaaaattggacataatggcaaaggaatcagtaaagcttacaggaggaaccataggaaacctaagaccagtaaacgcctccgaagtcctacaaatattcaaaaaatctgtgtaa

SEQ ID NO: 4 - protein, 390 aa, Unknown
  Description of Unknown Alcohol dehydrogenase polypeptide
MVDFEYSIPTRIFFGKDKINVLGRELKKYGSKVLIVYGGGSIKRNGIYDKAVSILEKNSIKFYELAGVEPNPRVTTVEKGVKICRENGVEVVLAIGGGSAIDCAKVIAAACEYDGNPWDIVLDGSKIKRVLPIASILTIAATGSEMDTWAVINNMDTNEKLIAAHPDMAPKFSILDPTYTYTVPTNQTAAGTADIMSHIFEVYFSNTKTAYLQDRMAEALLRTCIKYGGIALEKPDDYEARANLMWASSLAINGLLTYGKDTNWSVHLMEHELSAYYDITHGVGLAILTPNWMEYILNNDTVYKFVEYGVNVWGIDKEKNHYDIAHQAIQKTRDYFVNVLGLPSRLRDVGIEEEKLDIMAKESVKLTGGTIGNLRPVNASEVLQIFKKSV

SEQ ID NO: 5 - DNA, 2589 bp, Unknown
  Description of Unknown Bifunctional acetaldehyde/alcohol dehydrogenase polynucleotide
atgaaagtcacaacagtaaaggaattagatgaaaaactcaaggtaattaaagaagctcaaaaaaaattctcttgttactcgcaagaaatggttgatgaaatctttagaaatgcagcaatggcagcaatcgacgcaaggatagagctagcaaaagcagctgttttggaaaccggtatgggcttagttgaagacaaggttataaaaaatcattttgcaggcgaatacatctataacaaatataaggatgaaaaaacctgcggtataattgaacgaaatgaaccctacggaattacaaaaatagcagaacctataggagttgtagctgctataatccctgtaacaaaccccacatcaacaacaatatttaaatccttaatatcccttaaaactagaaatggaattttcttttcgcctcacccaagggcaaaaaaatccacaatactagcagctaaaacaatacttgatgcagccgttaagagtggtgccccggaaaatataataggttggatagatgaaccttcaattgaactaactcaatatttaatgcaaaaagcagatataacccttgcaactggtggtccctcactagttaaatctgcttattcttccggaaaaccagcaataggtgttggtccgggtaacaccccagtaataattgatgaatctgctcatataaaaatggcagtaagttcaattatattatccaaaacctatgataatggtgttatatgtgcttctgaacaatctgtaatagtcttaaaatccatatataacaaggtaaaagatgagttccaagaaagaggagcttatataataaagaaaaacgaattggataaagtccgtgaagtgatttttaaagatggatccgtaaaccctaaaatagtcggacagtcagcttatactatagcagctatggctggcataaaagtacctaaaaccacaagaatattaataggagaagttacctccttaggtgaagaagaaccttttgcccacgaaaaactatctcctgttttggctatgtatgaggctgacaattttgatgatgctttaaaaaaagcagtaactctaataaacttaggaggcctcggccatacctcaggaatatatgcagatgaaataaaagcacgagataaaatagatagatttagtagtgccatgaaaaccgtaagaacctttgtaaatatcccaacctcacaaggtgcaagtggagatctatataattttagaataccaccttctttcacgcttggctgcggattttggggaggaaattctgtttccgagaatgttggtccaaaacatcttttgaatattaaaaccgtagctgaaaggagagaaaacatgctttggtttagagttccacataaagtatattttaagttcggttgtcttcaatttgctttaaaagatttaaaagatctaaagaaaaaaagagcctttatagttactgatagtgacccctataatttaaactatgttgattcaataataaaaatacttgagcacctagatattgattttaaagtatttaataaggttggaagagaagctgatcttaaaaccataaaaaaagcaactgaagaaatgtcctcctttatgccagacactataatagctttaggtggtacccctgaaatgagctctgcaaagctaatgtgggtactatatgaacatccagaagtaaaatttgaagatcttgcaataaaatttatggacataagaaagagaatatatactttcccaaaactcggtaaaaaggctatgttagttgcaattacaacttctgctggttccggttctgaggttactccttttgctttagtaactgacaataacactggaaataagtacatgttagcagattatgaaatgacaccaaatatggcaattgtagatgcagaacttatgatgaaaatgccaaagggattaaccgcttattcaggtatagatgcactagtaaatagtatagaagcatacacatccgtatatgcttcagaatacacaaacggactagcactagaggcaatacgattaatatttaaatatttgcctgaggcttacaaaaacggaagaaccaatgaaaaagcaagagagaaaatggctcacgcttcaactatggcaggtatggcatccgctaatgcatttctaggtctatgtcattccatggcaataaaattaagttcagaacacaatattcctagtggcattgccaatgcattactaatagaagaagtaataaaatttaacgcagttgataatcctgtaaaacaagccccttgcccacaatataagtatccaaacaccatatttagatatgctcgaattgcagattatataaagcttggaggaaatactgatgaggaaaaggtagatctcttaattaacaaaatacatgaactaaaaaaagctttaaatataccaacttcaataaaggatgcaggtgttttggaggaaaacttctattcctcccttgatagaatatctgaacttgcactagatgatcaatgcacaggcgctaatcctagatttcctcttacaagtgagataaaagaaatgtatataaattgttttaaaaaacaaccttaa

SEQ ID NO: 6 - protein, 862 aa, Unknown
  Description of Unknown Bifunctional acetaldehyde/alcohol dehydrogenase polypeptide
MKVTTVKELDEKLKVIKEAQKKFSCYSQEMVDEIFRNAAMAAIDARIELAKAAVLETGMGLVEDKVIKNHFAGEYIYNKYKDEKTCGIIERNEPYGITKIAEPIGVVAAIIPVTNPTSTTIFKSLISLKTRNGIFFSPHPRAKKSTILAAKTILDAAVKSGAPENIIGWIDEPSIELTQYLMQKADITLATGGPSLVKSAYSSGKPAIGVGPGNTPVIIDESAHIKMAVSSIILSKTYDNGVICASEQSVIVLKSIYNKVKDEFQERGAYIIKKNELDKVREVIFKDGSVNPKIVGQSAYTIAAMAGIKVPKTTRILIGEVTSLGEEEPFAHEKLSPVLAMYEADNFDDALKKAVTLINLGGLGHTSGIYADEIKARDKIDRFSSAMKTVRTFVNIPTSQGASGDLYNFRIPPSFTLGCGFWGGNSVSENVGPKHLLNIKTVAERRENMLWFRVPHKVYFKFGCLQFALKDLKDLKKKRAFIVTDSDPYNLNYVDSIIKILEHLDIDFKVFNKVGREADLKTIKKATEEMSSFMPDTIIALGGTPEMSSAKLMWVLYEHPEVKFEDLAIKFMDIRKRIYTFPKLGKKAMLVAITTSAGSGSEVTPFALVTDNNTGNKYMLADYEMTPNMAIVDAELMMKMPKGLTAYSGIDALVNSIEAYTSVYASEYTNGLALEAIRLIFKYLPEAYKNGRTNEKAREKMAHASTMAGMASANAFLGLCHSMAIKLSSEHNIPSGIANALLIEEVIKFNAVDNPVKQAPCPQYKYPNTIFRYARIADYIKLGGNTDEEKVDLLINKIHELKKALNIPTSIKDAGVLEENFYSSLDRISELALDDQCTGANPRFPLTSEIKEMYINCFKKQP

SEQ ID NO: 7 - DNA, 1824 bp, Unknown
  Description of Unknown Aldehyde oxidoreductase polynucleotide
atgtacggatataagggtaaggtattaagaattaatctaagtagtaaaacttatatagtggaagaattgaaaattgacaaagctaaaaaatttataggtgcaagagggttaggcgtaaaaaccttatttgacgaagtagatccaaaggtagatccattatcacctgataacaaatttattatagcagcgggaccacttacaggtgcacctgttccaacaagcggaagattcatggtagttactaaatcacctttaacaggaactattgctattgcaaattcaggtggaaaatggggagcagaattcaaagcagctggatacgatatgataatcgttgaaggtaaatctgataaagaagtttatgtaaatatagtagatgataaagtagaatttagggatgcttctcatgtttggggaaaactaacagaagaaactacaaaaatgcttcaacaggaaacagattcgagagctaaggttttatgcataggaccagctggggaaaagttatcacttatggcagcagttatgaatgatgttgatagaacagcaggacgtggtggtgttggagctgttatgggttcaaagaacttaaaagctattgtagttaaaggaagcggaaaagtaaaattatttgatgaacaaaaagtgaaggaagtagcacttgagaaaacaaatattttaagaaaagatccagtagctggtggaggacttccaacatacggaacagctgtacttgttaatattataaatgaaaatggtgtacatccagtaaagaattttcaaaaatcttatacagatcaagcagataagatcagtggagaaactttaactaaagattgcttagttagaaaaaatccttgctataggtgtccaattgcctgtggaagatgggtaaaacttgatgatggaactgaatgtggaggaccagaatatgaaacattatggtcatttggatctgattgtgatgtatacgatataaatgctgtaaatacagcaaatatgttgtgtaatgaatatggattagataccattacagcaggatgtactattgcagcagctatggaactttatcaaagaggttatattaaggatgaagaaatagcagcagatggattgtcacttaattggggagatgctaagtccatggttgaatgggtaaagaaaatgggacttagagaaggatttggagacaagatggcagatggttcatacagactttgtgactcatacggtgtacctgagtattcaatgactgtaaaaaaacaggaacttccagcatatgacccaagaggaatacagggacatggtattacttatgctgttaacaataggggaggatgtcacattaagggatatatggtaagtcctgaaatacttggctatccagaaaaacttgatagacttgcagtggaaggaaaagcaggatatgctagagtattccatgatttaacagctgttatagattcacttggattatgtatttttacaacatttggtcttggtgcacaggattatgttgatatgtataatgcagtagttggtggagaattacatgatgtaaattctttaatgttagctggagatagaatatggactttagaaaaaatatttaacttaaaggcaggcatagatagttcacaggatactcttccaaagagattgcttgaagaacaaattccagaaggaccatcaaaaggagaagttcataagttagatgtactactacctgaatattattcagtacgtggatgggataaaaatggtattcctacagaggaaacgttaaagaaattaggattagatgaatacgtaggtaagctttag

SEQ ID NO: 8 - protein, 607 aa, Unknown
  Description of Unknown Aldehyde oxidoreductase polypeptide
MYGYKGKVLRINLSSKTYIVEELKIDKAKKFIGARGLGVKTLFDEVDPKVDPLSPDNKFIIAAGPLTGAPVPTSGRFMVVTKSPLTGTIAIANSGGKWGAEFKAAGYDMIIVEGKSDKEVYVNIVDDKVEFRDASHVWGKLTEETTKMLQQETDSRAKVLCIGPAGEKLSLMAAVMNDVDRTAGRGGVGAVMGSKNLKAIVVKGSGKVKLFDEQKVKEVALEKTNILRKDPVAGGGLPTYGTAVLVNIINENGVHPVKNFQKSYTDQADKISGETLTKDCLVRKNPCYRCPIACGRWVKLDDGTECGGPEYETLWSFGSDCDVYDINAVNTANMLCNEYGLDTITAGCTIAAAMELYQRGYIKDEEIAADGLSLNWGDAKSMVEWVKKMGLREGFGDKMADGSYRLCDSYGVPEYSMTVKKQELPAYDPRGIQGHGITYAVNNRGGCHIKGYMVSPEILGYPEKLDRLAVEGKAGYARVFHDLTAVIDSLGLCIFTTFGLGAQDYVDMYNAVVGGELHDVNSLMLAGDRIWTLEKIFNLKAGIDSSQDTLPKRLLEEQIPEGPSKGEVHKLDVLLPEYYSVRGWDKNGIPTEETLKKLGLDEYVGKL

SEQ ID NO: 9 - DNA, 906 bp, Unknown
  Description of Unknown Phosphotransacetylase polynucleotide
gtgattaagagttttaatgaaattatcatgaaggtaaagagcaaagaaatgaaaaaagttgctgttgctgtagcacaagacgagccagtacttgaagcagtaagagatgctaagaaaaatggtattgcagatgctattcttgttggagaccatgacgaaatcgtgtcaatcgcgcttaaaataggaatggatgtaaatgattttgaaatagtaaacgagcctaacgttaagaaagctgctttaaaggcagtagagcttgtatcaactggaaaagctgatatggtaatgaagggacttgtaaatacagcaactttcttaagatctgtattaaacaaagaagttggacttagaacaggaaaaactatgtctcacgttgcagtatttgaaactgagaaatttgatagactattatttttaacagatgttgctttcaatacttatcctgaattaaaggaaaaaattgatatagtaaacaattcagttaaggttgcacatgcaataggaattgaaaatccaaaggttgctccaatttgtgcagttgaggttataaaccctaaaatgccatcaacacttgatgcagcaatgctttcaaaaatgagtgacagaggacaaattaaaggttgtgtagttgacggacctttagcacttgatatagctttatcagaagaagcagcacatcataagggagtaacaggagaagttgctggaaaagctgatatcttcttaatgccaaacatagaaacaggaaatgtaatgtataagactttaacatatacaactgattcaaaaaatggaggaatcttagttggaacttctgcaccagttgttttaacttcaagagctgacagccatgaaacaaaaatgaactctatagcacttgcagctttagttgcaggcaataaataa

SEQ ID NO: 10 - protein, 301 aa, Unknown
  Description of Unknown Phosphotransacetylase polypeptide
MIKSFNEIIMKVKSKEMKKVAVAVAQDEPVLEAVRDAKKNGIADAILVGDHDEIVSIALKIGMDVNDFEIVNEPNVKKAALKAVELVSTGKADMVMKGLVNTATFLRSVLNKEVGLRTGKTMSHVAVFETEKFDRLLFLTDVAFNTYPELKEKIDIVNNSVKVAHAIGIENPKVAPICAVEVINPKMPSTLDAAMLSKMSDRGQIKGCVVDGPLALDIALSEEAAHHKGVTGEVAGKADIFLMPNIETGNVMYKTLTYTTDSKNGGILVGTSAPVVLTSRADSHETKMNSIALAALVAGNK

SEQ ID NO: 11 - DNA, 1068 bp, Unknown
  Description of Unknown Acetate kinase polynucleotide
atgtatagattactaataatcaatcctggctcgacctcaactaaaattggtatttatgacgatgaaaaagagatatttgagaagactttaagacattcagctgaagagatagaaaaatataacactatatttgatcaatttcaattcagaaagaatgtaattttagatgcgttaaaagaagcaaacatagaagtaagttctttaaatgctgtagttggaagaggcggactcttaaagccaatagtaagtggaacttatgcagtaaatcaaaaaatgcttgaagaccttaaagtaggagttcaaggtcagcatgcgtcaaatcttggtggaattattgcaaatgaaatagcaaaagaaataaatgttccagcatacatagttgatccagttgttgtggatgagcttgatgaagtttcaagaatatcaggaatggctgacattccaagaaaaagtatattccatgcattaaatcaaaaagcagttgctagaagatatgcaaaagaagttggaaaaaaatacgaagatcttaatttaatcgtagtccacatgggtggaggtacttcagtaggtactcataaagatggtagagtaatagaagttaataatacacttgatggagaaggtccattctcaccagaaagaagtggtggagttccaataggagatcttgtaagattgtgcttcagcaacaaatatacttatgaagaagtaatgaaaaagataaacggcaaaggcggagttgttagttacttaaatactatcgattttaaggctgtagttgataaagctcttgaaggagataagaaatgtgcacttatatatgaagctttcacattccaggtagcaaaagagataggaaaatgttcaaccgttttaaaaggaaatgtagatgcaataatcttaacaggcggaattgcgtacaacgagcatgtatgtaatgccatagaggatagagtaaaattcatagcacctgtagttagatatggtggagaagatgaacttcttgcacttgcagaaggtggacttagagttttaagaggagaagaaaaagctaaggaatacaaataa

SEQ ID NO: 12 - protein, 355 aa, Unknown
  Description of Unknown Acetate kinase polypeptide
MYRLLIINPGSTSTKIGIYDDEKEIFEKTLRHSAEEIEKYNTIFDQFQFRKNVILDALKEANIEVSSLNAVVGRGGLLKPIVSGTYAVNQKMLEDLKVGVQGQHASNLGGIIANEIAKEINVPAYIVDPVVVDELDEVSRISGMADIPRKSIFHALNQKAVARRYAKEVGKKYEDLNLIVVHMGGGTSVGTHKDGRVIEVNNTLDGEGPFSPERSGGVPIGDLVRLCFSNKYTYEEVMKKINGKGGVVSYLNTIDFKAVVDKALEGDKKCALIYEAFTFQVAKEIGKCSTVLKGNVDAIILTGGIAYNEHVCNAIEDRVKFIAPVVRYGGEDELLALAEGGLRVLRGEEKAKEYK

SEQ ID NO: 13 - DNA, 657 bp, Unknown
  Description of Unknown CoA-transferase subunit A polynucleotide
atgaactctaaaataattagatttgaaaatttaaggtcattctttaaagatgggatgacaattatgattggaggttttttaaactgtggcactccaaccaaattaattgattttttagttaatttaaatataaagaatttaacgattataagtaatgatacatgttatcctaatacaggtattggtaagttaatatcaaataatcaagtaaaaaagcttattgcttcatatataggcagcaacccagatactggcaaaaaactttttaataatgaacttgaagtagagctctctccccaaggaactctagtggaaagaatacgtgcaggcggatctggcttaggtggtgtactaactaaaacaggtttaggaactttgattgaaaaaggaaagaaaaaaatatctataaatggaacggaatatttgttagagctacctcttacagccgatgtagcattaattaaaggtagtattgtagatgaggccggaaacaccttctataaaggtactactaaaaactttaatccctatatggcaatggcagctaaaaccgtaatagttgaagctgaaaatttagttagctgtgaaaaactagaaaaggaaaaagcaatgacccccggagttcttataaattatatagtaaaggagcctgcataa

SEQ ID NO: 14 - protein, 218 aa, Unknown
  Description of Unknown CoA-transferase subunit A polypeptide
MNSKIIRFENLRSFFKDGMTIMIGGFLNCGTPTKLIDFLVNLNIKNLTIISNDTCYPNTGIGKLISNNQVKKLIASYIGSNPDTGKKLFNNELEVELSPQGTLVERIRAGGSGLGGVLTKTGLGTLIEKGKKKISINGTEYLLELPLTADVALIKGSIVDEAGNTFYKGTTKNFNPYMAMAAKTVIVEAENLVSCEKLEKEKAMTPGVLINYIVKEPA

SEQ ID NO: 15 - DNA, 666 bp, Unknown
  Description of Unknown CoA-transferase subunit B polynucleotide
atgattaatgataaaaacctagcgaaagaaataatagccaaaagagttgcaagagaattaaaaaatggtcaacttgtaaacttaggtgtaggtcttcctaccatggttgcagattatataccaaaaaatttcaaaattactttccaatcagaaaacggaatagttggaatgggcgctagtcctaaaataaatgaggcagataaagatgtagtaaatgcaggaggagactatacaacagtacttcctgacggcacatttttcgatagctcagtttcgttttcactaatccgtggtggtcacgtagatgttactgttttaggggctctccaggtagatgaaaagggtaatatagccaattggattgttcctggaaaaatgctctctggtatgggtggagctatggatttagtaaatggagctaagaaagtaataattgcaatgagacatacaaataaaggtcaacctaaaattttaaaaaaatgtacacttcccctcacggcaaagtctcaagcaaatctaattgtaacagaacttggagtaattgaggttattaatgatggtttacttctcactgaaattaataaaaacacaaccattgatgaaataaggtctttaactgctgcagatttactcatatccaatgaacttagacccatggctgtttag

SEQ ID NO: 16 - protein, 221 aa, Unknown
  Description of Unknown CoA-transferase subunit B polypeptide
MINDKNLAKEIIAKRVARELKNGQLVNLGVGLPTMVADYIPKNFKITFQSENGIVGMGASPKINEADKDVVNAGGDYTTVLPDGTFFDSSVSFSLIRGGHVDVTVLGALQVDEKGNIANWIVPGKMLSGMGGAMDLVNGAKKVIIAMRHTNKGQPKILKKCTLPLTAKSQANLIVTELGVIEVINDGLLLTEINKNTTIDEIRSLTAADLLISNELRPMAV

SEQ ID NO: 17 - DNA, 735 bp, Unknown
  Description of Unknown Acetoacetate decarboxylase polynucleotide
atgttaaaggatgaagtaattaaacaaattagcacgccattaacttcgcctgcatttcctagaggaccctataaatttcataatcgtgagtattttaacattgtatatcgtacagatatggatgcacttcgtaaagttgtgccagagcctttagaaattgatgagcccttagtcaggtttgaaattatggcaatgcatgatacgagtggacttggttgttatacagaaagcggacaggctattcccgtaagctttaatggagttaagggagattatcttcatatgatgtatttagataatgagcctgcaattgcagtaggaagggaattaagtgcatatcctaaaaagctcgggtatccaaagctttttgtggattcagatactttagtaggaactttagactatggaaaacttagagttgcgacagctacaatggggtacaaacataaagccttagatgctaatgaagcaaaggatcaaatttgtcgccctaattatatgttgaaaataatacccaattatgatggaagccctagaatatgtgagcttataaatgcgaaaatcacagatgttaccgtacatgaagcttggacaggaccaactcgactgcagttatttgatcacgctatggcgccacttaatgatttgccagtaaaagagattgtttctagctctcacattcttgcagatataatattgcctagagctgaagttatatatgattatcttaagtaa

SEQ ID NO: 18 - protein, 244 aa, Unknown
  Description of Unknown Acetoacetate decarboxylase polypeptide
MLKDEVIKQISTPLTSPAFPRGPYKFHNREYFNIVYRTDMDALRKVVPEPLEIDEPLVRFEIMAMHDTSGLGCYTESGQAIPVSFNGVKGDYLHMMYLDNEPAIAVGRELSAYPKKLGYPKLFVDSDTLVGTLDYGKLRVATATMGYKHKALDANEAKDQICRPNYMLKIIPNYDGSPRICELINAKITDVTVHEAWTGPTRLQLFDHAMAPLNDLPVKEIVSSSHILADIILPRAEVIYDYLK

SEQ ID NO: 19 - DNA, 1056 bp, Unknown
  Description of Unknown Secondary alcohol dehydrogenase polynucleotide
atgaaaggttttgcaatgttaggtattaacaaattaggatggattgaaaagaaaaacccagtgccaggtccttatgatgcgattgtacatcctctagctgtatccccatgtacatcagatatacatacggtttttgaaggagcacttggtaatagggaaaatatgattttaggccatgaagctgtaggtgaaatagccgaagttggcagcgaagttaaagattttaaagttggcgatagagttatcgtaccatgcacaacacctgactggagatctttagaagtccaagctggttttcagcagcattcaaacggtatgcttgcaggatggaagttttccaattttaaagatggtgtatttgcagattactttcatgtaaacgatgcagatatgaatcttgccatactcccagatgaaatacctttagaaagtgcagttatgatgacagacatgatgactactggttttcatggagcagaacttgcagacataaaaatgggctccagcgttgtagtaattggtataggagctgttggattaatgggaatagccggttccaaacttcgaggagcaggcagaattatcggtgttggaagcagacctgtttgtgttgaaacagctaaattttatggagcaactgatattgtaaattataaaaatggtgatatagttgaacaaatcatggacttaactcatggtaaaggtgtagaccgtgtaatcatggcaggcggtggtgctgaaacactagcacaagcagtaactatggttaaacctggcggcgtaatttctaacatcaactaccatggaagcggtgatactttaccaatacctcgtgttcaatggggctgcggcatggctcacaaaactataagaggaggattatgccccggcggacgtcttagaatggaaatgctaagagatcttgttctatataaacgtgttgatttgagtaaacttgttactcatgtatttgatggtgcagaaaatattgaaaaggcccttttgcttatgaaaaataagccaaaagatttaattaaatcagtagttacattctaa

SEQ ID NO: 20 - protein, 351 aa, Unknown
  Description of Unknown Secondary alcohol dehydrogenase polypeptide
MKGFAMLGINKLGWIEKKNPVPGPYDAIVHPLAVSPCTSDIHTVFEGALGNRENMILGHEAVGEIAEVGSEVKDFKVGDRVIVPCTTPDWRSLEVQAGFQQHSNGMLAGWKFSNFKDGVFADYFHVNDADMNLAILPDEIPLESAVMMTDMMTTGFHGAELADIKMGSSVVVIGIGAVGLMGIAGSKLRGAGRIIGVGSRPVCVETAKFYGATDIVNYKNGDIVEQIMDLTHGKGVDRVIMAGGGAETLAQAVTMVKPGGVISNINYHGSGDTLPIPRVQWGCGMAHKTIRGGLCPGGRLRMEMLRDLVLYKRVDLSKLVTHVFDGAENIEKALLLMKNKPKDLIKSVVTF

SEQ ID NO: 21 - DNA, 1140 bp, Unknown
  Description of Unknown Butyryl-CoA dehydrogenase polynucleotide
atggattttaatttaacaagagaacaagaattagtaagacagatggttagagaatttgctgaaaatgaagttaaacctatagcagcagaaattgatgaaacagaaagatttccaatggaaaatgtaaagaaaatgggtcagtatggtatgatgggaattccattttcaaaagagtatggtggcgcaggtggagatgtattatcttatataatcgccgttgaggaattatcaaaggtttgcggtactacaggagttattctttcagcacatacatcactttgtgcttcattaataaatgaacatggtacagaagaacaaaaacaaaaatatttagtacctttagctaaaggtgaaaaaataggtgcttatggattgactgagccaaatgcaggaacagattctggagcacaacaaacagtagctgtacttgaaggagatcattatgtaattaatggttcaaaaatattcataactaatggaggagttgcagatacttttgttatatttgcaatgactgacagaactaaaggaacaaaaggtatatcagcatttataatagaaaaaggcttcaaaggtttctctattggtaaagttgaacaaaagcttggaataagagcttcatcaacaactgaacttgtatttgaagatatgatagtaccagtagaaaacatgattggtaaagaaggaaaaggcttccctatagcaatgaaaactcttgatggaggaagaattggtatagcagctcaagctttaggtatagctgaaggtgctttcaacgaagcaagagcttacatgaaggagagaaaacaatttggaagaagccttgacaaattccaaggtcttgcatggatgatggcagatatggatgtagctatagaatcagctagatatttagtatataaagcagcatatcttaaacaagcaggacttccatacacagttgatgctgcaagagctaagcttcatgctgcaaatgtagcaatggatgtaacaactaaggcagtacaattatttggtggatacggatatacaaaagattatccagttgaaagaatgatgagagatgctaagataactgaaatatatgaaggaacttcagaagttcagaaattagttatttcaggaaaaatttttagataa

SEQ ID NO: 22 - protein, 379 aa, Unknown
  Description of Unknown Butyryl-CoA dehydrogenase polypeptide
MDFNLTREQELVRQMVREFAENEVKPIAAEIDETERFPMENVKKMGQYGMMGIPFSKEYGGAGGDVLSYIIAVEELSKVCGTTGVILSAHTSLCASLINEHGTEEQKQKYLVPLAKGEKIGAYGLTEPNAGTDSGAQQTVAVLEGDHYVINGSKIFITNGGVADTFVIFAMTDRTKGTKGISAFIIEKGFKGFSIGKVEQKLGIRASSTTELVFEDMIVPVENMIGKEGKGFPIAMKTLDGGRIGIAAQALGIAEGAFNEARAYMKERKQFGRSLDKFQGLAWMMADMDVAIESARYLVYKAAYLKQAGLPYTVDAARAKLHAANVAMDVTTKAVQLFGGYGYTKDYPVERMMRDAKITEIYEGTSEVQKLVISGKIFR

SEQ ID NO: 23 - DNA, 1197 bp, Unknown
  Description of Unknown Trans-2-enoyl-CoA reductase polynucleotide
atgatagtaaaagcaaagtttgtaaaaggatttatcagagatgtacatccttatggttgcagaagggaagtactaaatcaaatagattattgtaagaaggctattgggtttaggggaccaaagaaggttttaattgttggagcctcatctgggtttggtcttgctactagaatttcagttgcatttggaggtccagaagctcacacaattggagtatcctatgaaacaggagctacagatagaagaataggaacagcgggatggtataataacatattttttaaagaatttgctaaaaaaaaaggattagttgcaaaaaacttcattgaggatgccttttctaatgaaaccaaagataaagttattaagtatataaaggatgaatttggtaaaatagatttatttgtttatagtttagctgcgcctaggagaaaggactataaaactggaaatgtttatacttcaagaataaaaacaattttaggagattttgagggaccgactattgatgttgaaagagacgagattactttaaaaaaggttagtagtgctagcattgaagaaattgaagaaactagaaaggtaatgggtggagaggattggcaagagtggtgtgaagagctgctttatgaagattgtttttcggataaagcaactaccatagcatactcgtatataggatccccaagaacctacaagatatatagagaaggtactataggaatagctaaaaaggatcttgaagataaggctaagcttataaatgaaaaacttaacagagttataggtggtagagcctttgtgtctgtgaataaagcattagttacaaaagcaagtgcatatattccaacttttcctctttatgcagctattttatataaggtcatgaaagaaaaaaatattcatgaaaattgtattatgcaaattgagagaatgttttctgaaaaaatatattcaaatgaaaaaatacaatttgatgacaagggaagattaaggatggacgatttagagcttagaaaagacgttcaagacgaagttgatagaatatggagtaatattactcctgaaaattttaaggaattatctgattataagggatacaaaaaagaattcatgaacttaaacggttttgatctagatggggttgattatagtaaagacctggatatagaattattaagaaaattagaaccttaa

SEQ ID NO: 24 - protein, 398 aa, Unknown
  Description of Unknown Trans-2-enoyl-CoA reductase polypeptide
MIVKAKFVKGFIRDVHPYGCRREVLNQIDYCKKAIGFRGPKKVLIVGASSGFGLATRISVAFGGPEAHTIGVSYETGATDRRIGTAGWYNNIFFKEFAKKKGLVAKNFIEDAFSNETKDKVIKYIKDEFGKIDLFVYSLAAPRRKDYKTGNVYTSRIKTILGDFEGPTIDVERDEITLKKVSSASIEEIEETRKVMGGEDWQEWCEELLYEDCFSDKATTIAYSYIGSPRTYKIYREGTIGIAKKDLEDKAKLINEKLNRVIGGRAFVSVNKALVTKASAYIPTFPLYAAILYKVMKEKNIHENCIMQIERMFSEKIYSNEKIQFDDKGRLRMDDLELRKDVQDEVDRIWSNITPENFKELSDYKGYKKEFMNLNGFDLDGVDYSKDLDIELLRKLEP

(19) United States
(12) Patent Application Publication
(10) Pub. No.: US 2019/0218578 A1
(43) Pub. Date: Jul. 18, 2019

(54) MICROBIAL ORGANISMS FOR CONVERTING ACETYL-COA INTO CROTYL ALCOHOL AND METHODS FOR PRODUCING CROTYL ALCOHOL

(71) Applicant: White Dog Labs, Inc., New Castle, DE (US)

(72) Inventors: Bryan P. TRACY, Wilmington, DE (US); Aharon M. EYAL, Jerusalem (IL); Shawn William JONES, Bear, DE (US); Carrissa A. WIEDEL, Bel Air, MD (US)

(73) Assignee: White Dog Labs, Inc., New Castle, DE (US)

(21) Appl. No.: 15/754,937

(22) PCT Filed: Aug. 23, 2016
(86) PCT No.: PCT/US16/48197

§ 371 Date: Feb. 23, 2018

Related U.S. Application Data
(60) Provisional application No. 62/209,133, filed on Aug. 24, 2015.

(51) Int. Cl. C12P 7/28; C12P 7/56; C12N 9/04; C12P 7/04; C12N 9/02; C12N 9/12; C12N 9/10

(52) U.S. Cl. CPC C12P 7/28; C12P 7/56; C12N 9/0006; C12P 7/04; C12N 9/0008; C12N 9/1217; C12Y 102/07005; C12Y 102/01057; C12Y 101/01001; C12Y 203/01019; C12Y 102/0101; C12Y 207/02007; C12N 9/1029

(57) ABSTRACT

The present invention provides microorganisms capable of converting acetyl-coA into crotyl alcohol as well as fermentation methods for producing crotyl alcohol, either alone, or in combination with acetone and/or isopropanol. The microorganisms may be genetically engineered to express and/or disrupt one or more of the following enzymes: acetaldehyde dehydrogenase, alcohol dehydrogenase, bifunctional acetaldehyde/alcohol dehydrogenase, aldehyde oxidoreductase, phosphotransacetylase, acetate kinase, CoA-transferase A, CoA-transferase B, acetoacetate decarboxylase, secondary alcohol dehydrogenase, butyryl-CoA dehydro genase (BCD), and/or trans-2-enoyl-CoA reductase (TER).

### CROSS-REFERENCE TO RELATED APPLICATION

The present application claims the benefit under 35 U.S.C. § 119(e) of U.S. Provisional Application No. 62/209,133 filed Aug. 24, 2015, the disclosure of which is expressly incorporated by reference herein in its entirety..

### FIELD OF THE INVENTION

The present invention involves the fermentative production of organic products such as crotyl alcohol, acetone, and isopropanol, as well as microorganisms capable of converting acetyl-CoA into crotyl alcohol.

### BACKGROUND

Crotyl alcohol has historically been of little commercial interest and overlooked as a biosynthetic/fermentation production endpoint. Efforts have instead focused on fermentative production of downstream targets such as butadiene and/or intermediates such as acetyl-CoA.

More recently, production of crotyl alcohol has garnered some attention in the fields of plastics, agriculture, and pharmaceuticals, primarily as an intermediate to make 1,3-butadiene. For example, U.S. Pat. No. 9,169,496 describes enzymatic production of butadiene from crotyl alcohol but fails to teach production of crotyl alcohol in a genetically modified organism, much less as a production endpoint.

U.S. Pat. No. 8,580,543, U.S. Pat. No. 9,169,486, and U.S. Pat. No. 9,321,701 describe genetically modified microbial organisms as well as methods for production of butadiene via a crotyl alcohol intermediate. However, the genetically modified microorganisms lack an endogenous ability to convert acetyl-CoA to crotonyl-CoA, much less to crotyl alcohol. Additionally, crotyl alcohol is only considered as an intermediate product formed in the production of the target bioproduct: 1,3-butadiene.

Thus, there remains a need for efficient and cost-effective methods for producing crotyl alcohol, and for engineered microbial organisms capable of producing high quantities of crotyl alcohol.

### SUMMARY OF THE INVENTION

Provided herein is a non-naturally occurring microbial organism capable of converting acetyl-CoA into crotyl alcohol, wherein at least one of the following genes are deleted, disrupted or silenced and/or expression from at least one of the following genes is disrupted or silenced:

i. Butyryl-CoA dehydrogenase (BDC); and/or

ii. Trans-2-enoyl-CoA reductase (TER).

In an embodiment, said microbial organism comprises a disrupted, deleted, or mutated BCD and/or TER gene. In an embodiment, said disruption or silencing of expression includes disruption or silencing of RNA transcription and/or protein translation. In an embodiment, disruption or silencing of expression includes protein translation silencing using RNA interference. In an embodiment, said microbial organism produces more crotyl alcohol compared with a naturally occurring microbial organism of the same genus and species lacking said disrupted, deleted, or silenced BCD gene and/or said disrupted, deleted or silenced TER gene.

In an embodiment, said microbial organism comprises at least one exogenous nucleic acid encoding one or more of the following enzymes for producing crotyl alcohol from crotonyl-CoA:

A. Acetaldehyde dehydrogenase;

B. Alcohol dehydrogenase;

C. Bifunctional acetaldehyde/alcohol dehydrogenase;

D. Aldehyde oxidoreductase;

E. Phosphotransacetylase; and/or

F. Acetate kinase.

In an embodiment, said microbial organism is capable of further producing acetone and comprises at least a second exogenous nucleic acid encoding one or more acetone pathway enzymes. In an embodiment, said one or more acetone pathway enzymes comprises:

G. CoA-transferase subunit A;

H. CoA-transferase subunit B; and/or

I. Acetoacetate decarboxylase.

In an embodiment, said microbial organism is capable of further producing isopropanol and comprises at least a second exogenous nucleic acid encoding one or more isopropanol pathway enzymes. In an embodiment, said one or more isopropanol pathway enzymes comprises:

G. CoA-transferase subunit A;

H. CoA-transferase subunit B;

I. Acetoacetate decarboxylase; and/or

J. Secondary alcohol dehydrogenase.

In an embodiment, said microbial organism comprises exogenous nucleic acids encoding each of the enzymes A, B, G, H, and I. In an embodiment, said microbial organism comprises exogenous nucleic acids encoding each of the enzymes C, G, H, and I. In an embodiment, said microbial organism comprises exogenous nucleic acids encoding each of the enzymes B, D, E, F, G, H, and I. In an embodiment, said microbial organism comprises exogenous nucleic acids encoding each of the enzymes C, D, E, F, G, H, and I. In an embodiment, said microbial organism comprises exogenous nucleic acids encoding each of the enzymes A, B, C, G, H, and I. In an embodiment, said microbial organism comprises exogenous nucleic acids encoding each of the enzymes A, B, D, E, F, G, H, and I. In an embodiment, said microbial organism comprises exogenous nucleic acids encoding each of the enzymes A, B, C, D, E, F, G, H, and I.

In an embodiment, said microbial organism comprises exogenous nucleic acids encoding each of the enzymes A, B, G, H, I, and J. In an embodiment, said microbial organism comprises exogenous nucleic acids encoding each of the enzymes C, G, H, I, and J. In an embodiment, said microbial organism comprises exogenous nucleic acids encoding each of the enzymes B, D, E, F, G, H, I, and J. In an embodiment, said microbial organism comprises exogenous nucleic acids encoding each of the enzymes C, D, E, F, G, H, I, and J. In an embodiment, said microbial organism comprises exogenous nucleic acids encoding each of the enzymes A, B, C, G, H, I, and J. In an embodiment, said microbial organism comprises exogenous nucleic acids encoding each of the enzymes A, B, D, E, F, G, H, I, and J. In an embodiment, said microbial organism comprises exogenous nucleic acids encoding each of the enzymes A, B, C, D, E, F, G, H, I, and J.

In an embodiment, a microbial organism as provided herein may comprise two, three, four, five, six, seven, eight, nine, or ten exogenous nucleic acids.

Also provided herein is such a microbial organism, wherein the exogenous nucleic acid is a heterologous nucleic acid.

Also provided herein is such a microbial organism, wherein said organism is an acetogenic bacterium.

Herein is also provided a method of producing crotyl alcohol, comprising culturing said microbial organism as above on a growth substrate, under conditions to form a broth comprising crotyl alcohol. Also provided is a method of producing crotyl alcohol and acetone, comprising culturing said microbial organism on a growth substrate, under conditions to form a broth comprising crotyl alcohol and acetone. In an embodiment, the acetone to crotyl alcohol molar ratio in said broth is in the range from 0.1 to 0.95. Also provided is a method of producing crotyl alcohol and isopropanol, comprising culturing said microbial organism on a growth substrate, under conditions to form a broth comprising crotyl alcohol and isopropanol. In an embodiment, the isopropanol to crotyl alcohol molar ratio in said broth is in the range from 0.1 to 0.95.

Also provided is such a method, wherein said growth substrate comprises a carbohydrate.

Also provided is such a method, wherein said growth substrate further comprises a one-carbon molecule. In an embodiment, such a method may be performed, wherein said one-carbon molecule is exogenously added. In an embodiment, said one-carbon molecule may be selected from the group consisting of CO, CO₂, CH₃OH, carbonate, bicarbonate, urea, and combinations thereof.

Also provided is such a method, wherein said growth substrate comprises at least one gaseous compound. In an embodiment, said gaseous compound is exogenously added. In an embodiment, said at least one gaseous compound is selected from a group consisting of CO, CO₂, H₂ and combinations thereof.

Also provided herein is such a method, wherein said growth substrate comprises a carbohydrate in combination with at least one of a one-carbon molecule and a gaseous compound.

Also provided herein is such a method, wherein said growth substrate comprises a carbohydrate, exogenously added CO₂ and exogenously added H₂, and wherein at least 2 moles of H₂ are added per mole of CO₂.

Also provided herein is such a method, comprising steam reforming of a hydrocarbon, whereby a synthesis gas comprising CO, CO₂ and H₂ is produced and the synthesis gas forms a part of said growth substrate.

Also provided herein is such a method, comprising supplementing pressurized CO₂, pressurized CO, pressurized H₂, or a combination thereof to said growth substrate.

Also provided herein is such a method, wherein said culturing is conducted at a pressure in the range between 1 atm and 5 atm.

Also provided herein is such a method, comprising supplementing at least one of ammonium carbonate and ammonium bicarbonate to said growth substrate.

In an embodiment, the method may comprise supplementing pressurized CO₂ to said growth substrate.

Also provided herein is such a method, comprising at least partially separating crotyl alcohol from said broth to form separated crotyl alcohol.

Also provided herein is such a method, comprising at least partially separating acetone from said broth.

Also provided herein is such a method, comprising at least partially separating isopropanol from said broth.

Also provided herein is such a method, wherein said separating comprises liquid-liquid extraction. In an embodiment, the method may further comprise dehydrating said separated crotyl alcohol to form butadiene.

Provided herein is a microbial organism capable of naturally converting acetyl-CoA into crotonyl-CoA, the microbial organism comprising at least one exogenous nucleic acid encoding one or more of the following crotyl alcohol pathway enzymes:

A. Acetaldehyde dehydrogenase;

B. Alcohol dehydrogenase;

C. Bifunctional acetaldehyde/alcohol dehydrogenase;

D. Aldehyde oxidoreductase;

E. Phosphotransacetylase; and/or

F. Acetate kinase,

wherein said microbial organism produced more crotyl alcohol compared with a naturally occurring microbial organism of the same genus and species lacking said exogenous nucleic acid.

In an embodiment, the expression of butyryl-CoA dehydrogenase (BCD) in said microbial organism is disrupted or silenced. In an embodiment, the microbial organism comprises a disrupted, deleted, or mutated BCD gene. In an embodiment, the protein translation of BCD is silenced using RNA interference.

In an embodiment, the expression of trans-2-enoyl-CoA reductase (TER) in said microbial organism is disrupted or silenced. In an embodiment, the microbial organism comprises a disrupted, deleted, or mutated TER gene. In an embodiment, the protein translation of TER is silenced using RNA interference.

In an embodiment, the expression of both butyryl-CoA dehydrogenase (BCD) and trans-2-enoyl-CoA reductase (TER) in said microbial organism are disrupted or silenced. In an embodiment, the microbial organism comprises a disrupted, deleted, or mutated BCD gene and a disrupted, deleted, or mutated TER gene. In an embodiment, the protein translation of BCD and TER are silenced using RNA interference.

In an embodiment, said microbial organism is capable of further producing acetone and comprises at least a second exogenous nucleic acid encoding one or more acetone pathway enzymes. In an embodiment, said one or more acetone pathway enzymes comprises:

J. CoA-transferase subunit A;

K. CoA-transferase subunit B; and/or

L. Acetoacetate decarboxylase.

In an embodiment, said microbial organism is capable of further producing isopropanol and comprises at least a second exogenous nucleic acid encoding one or more isopropanol pathway enzymes. In an embodiment, said one or more isopropanol pathway enzymes comprises:

G. CoA-transferase subunit A;

H. CoA-transferase subunit B;

I. Acetoacetate decarboxylase; and/or

J. Secondary alcohol dehydrogenase.

In an embodiment, said microbial organism capable of further producing acetone comprises exogenous nucleic acids encoding each of the enzymes A, B, G, H, and I. In an embodiment, said microbial organism comprises exogenous nucleic acids encoding each of the enzymes C, G, H, and I. In an embodiment, said microbial organism comprises exogenous nucleic acids encoding each of the enzymes B, D, E, F, G, H, and I. In an embodiment, said microbial organism comprises exogenous nucleic acids encoding each of the enzymes C, D, E, F, G, H, and I. In an embodiment, said microbial organism comprises exogenous nucleic acids encoding each of the enzymes A, B, C, G, H, and I. In an embodiment, said microbial organism comprises exogenous nucleic acids encoding each of the enzymes A, B, D, E, F, G, H, and I. In an embodiment, said microbial organism comprises exogenous nucleic acids encoding each of the enzymes A, B, C, D, E, F, G, H, and I.

In an embodiment, said microbial organism capable of further producing isopropanol comprises exogenous nucleic acids encoding each of the enzymes A, B, G, H, I, and J. In an embodiment, said microbial organism comprises exogenous nucleic acids encoding each of the enzymes C, G, H, I, and J. In an embodiment, said microbial organism comprises exogenous nucleic acids encoding each of the enzymes B, D, E, F, G, H, I, and J. In an embodiment, said microbial organism comprises exogenous nucleic acids encoding each of the enzymes C, D, E, F, G, H, I, and J. In an embodiment, said microbial organism comprises exogenous nucleic acids encoding each of the enzymes A, B, C, G, H, I, and J. In an embodiment, said microbial organism comprises exogenous nucleic acids encoding each of the enzymes A, B, D, E, F, G, H, I, and J. In an embodiment, said microbial organism comprises exogenous nucleic acids encoding each of the enzymes A, B, C, D, E, F, G, H, I, and J.

In an embodiment, a microbial organism as provided herein may comprise two, three, four, five, six, seven, eight, nine, or ten exogenous nucleic acids.

Also provided herein is such a microbial organism, wherein the exogenous nucleic acid is a heterologous nucleic acid.

Also provided herein is such a microbial organism, wherein said organism is an acetogenic bacterium.

Herein is also provided a method of producing crotyl alcohol, comprising culturing said microbial organism on a growth substrate, under conditions to form a broth comprising crotyl alcohol. Also provided is a method of producing crotyl alcohol and acetone, comprising culturing said microbial organism on a growth substrate, under conditions to form a broth comprising crotyl alcohol and acetone. In an embodiment, the acetone to crotyl alcohol molar ratio in said broth is in the range from 0.1 to 0.95. Also provided is a method of producing crotyl alcohol and isopropanol, comprising culturing said microbial organism on a growth substrate, under conditions to form a broth comprising crotyl alcohol and isopropanol. In an embodiment, the isopropanol to crotyl alcohol molar ratio in said broth is in the range from 0.1 to 0.95.

Also provided is such a method, wherein said growth substrate comprises a carbohydrate.

Also provided is such a method, wherein said growth substrate further comprises a one-carbon molecule. In an embodiment, such a method may be performed, wherein said one-carbon molecule is exogenously added. In an embodiment, said one-carbon molecule is selected from the group consisting of CO, CO₂, CH₃OH, carbonate, bicarbonate, urea, and combinations thereof.

Also provided is such a method, wherein said growth substrate comprises at least one gaseous compound. In an embodiment, said at least one gaseous compound is exogenously added. In an embodiment, said at least one gaseous compound is selected from the group consisting of CO, CO₂, H₂ and combinations thereof.

Also provided herein is such a method, wherein said growth substrate comprises a carbohydrate in combination with at least one of a one-carbon molecule and a gaseous compound.

Also provided herein is such a method, wherein said growth substrate comprises a carbohydrate, exogenously added CO₂ and exogenously added H₂, and wherein at least 2 moles of H₂ are added per mole of CO₂.

Also provided herein is such a method, comprising steam reforming of a hydrocarbon, whereby a synthesis gas comprising CO, CO₂ and H₂ is produced and the synthesis gas forms a part of said growth substrate.

Also provided herein is such a method, comprising supplementing pressurized CO₂, pressurized CO, pressurized H₂, or a combination thereof to said growth substrate.

Also provided herein is such a method, wherein said culturing is conducted at a pressure in the range between 1 atm and 5 atm.

Also provided herein is such a method, comprising supplementing at least one of ammonium carbonate and ammonium bicarbonate to said growth substrate.

In an embodiment, the method may comprise supplementing pressurized CO₂ to said growth substrate.

Also provided herein is such a method, comprising at least partially separating crotyl alcohol from said broth to form separated crotyl alcohol.

Also provided herein is such a method, comprising at least partially separating acetone from said broth.

Also provided herein is such a method, comprising at least partially separating isopropanol from said broth.

Also provided herein is such a method, wherein said separating comprises liquid-liquid extraction. In an embodiment, the method may further comprise dehydrating said separated crotyl alcohol to form butadiene.

### DETAILED DESCRIPTION OF THE INVENTION

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

As used herein, the term “about”, when referring to a measurable value such as an amount, a temporal duration, and the like, is meant to encompass variations of +/−10%, more preferably +/−5%, even more preferably, +/−1%, and still more preferably +/−0.1% from the specified value, as such variations are appropriate to perform the disclosed methods.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

The presently claimed subject matter relates to novel microorganisms and biosynthesis methods for production of crotyl alcohol, acetone, and isopropanol. Unexpectedly superior levels of crotyl alcohol, acetone, and/or isopropanol production levels are achieved with microorganisms as described herein and by methods of their use.

### I. MICROORGANISMS OF THE INVENTION

Microorganisms suitable for use in the present invention are not particularly limited as long as the native form of the microorganisms is capable of converting acetyl-CoA into crotonyl-CoA.

Host organisms suitable for use in the invention include bacteria, including acetogenic bacteria, yeast, fungi and/or other microorganisms known for use in fermentative processes.

Example organisms that are naturally capable of converting acetyl-CoA into crotonyl-CoA include bacteria such as Clostridium acetobutylicum, Clostridium beijerinckii, Clostridium kluyveri, Clostridium saccharoperbutylacetonicum, Clostridium pasteurianum, Clostridium saccharobutylicum, Clostridium carboxidovorans, Clostridium butyricum, Clostridium tyrobutyricum, Clostridium cellulovorans, Clostridium bornimense, Clostridium scatologenes, Clostridium drakei, Clostridium tetani, Clostridium baratii, Clostridium perfringens, Clostridium botulinum, Clostridium novyi, Clostridium sporogenes, Clostridium sticklandii, Thermoanaerobacterium thermosaccharolyticum, Fervidobacterium pennivorans, Fervidobacterium nodosum, Thermoanaerobacter wiegelii, Thermoanaerobacter tengcongensis, Alkaliphilus metalliredigens, Alkaliphilus oremlandii, Eubacterium limosum, Eubacterium aggregans, Butyribacterium methylotrophicum, Peptoclostridium difficile, and Oxobacter pfennigii.

In an embodiment, the microorganism may be genetically modified to express one or more of the following crotyl alcohol pathway enzymes: acetaldehyde dehydrogenase (aldehyde forming enzyme), alcohol dehydrogenase (alcohol forming enzyme), bifunctional acetaldehyde/alcohol dehydrogenase (aldehyde & alcohol forming enzyme), aldehyde oxidoreductase (aldehyde forming enzyme), phosphotransacetylase (phosphate forming enzyme), and/or acetate kinase (carboxylic acid forming enzyme).

In an embodiment, the microorganism may be genetically modified to express one or more of the following acetone pathway enzymes: CoA-transferase subunit A, CoA-transferase subunit B, and/or acetoacetate decarboxylase.

In an embodiment, the microorganism may be genetically modified to express one or more of the following isopropanol pathway enzymes: CoA-transferase subunit A, CoA-transferase subunit B, acetoacetate decarboxylase, and/or secondary alcohol dehydrogenase.

In an embodiment, the microorganism may have decreased expression of butyryl-CoA dehydrogenase (BCD) or BCD expression may be silenced.

In an embodiment, the microorganism may have decreased expression of trans-2-enoyl-CoA reductase (TER) or TER expression may be silenced.

Of course, the above genetic modifications are not particularly limited and one or more genes may be inserted into the genome of the host microorganism in combination. Additionally, one or more genes may be disrupted or silenced while others have increased expression.

Depending on the host microorganism selected for production of crotyl alcohol, acetone, and/or isopropanol, nucleic acids for some or all of a particular biosynthetic pathway can be expressed. For example, if a selected microorganism is deficient in a desired biosynthetic pathway, then exogenous nucleic acids encoding the enzymes for the desired pathway may be introduced into the microbial host. Alternatively, if the selected microorganism expresses some pathway enzymes/genes, but is deficient in others, an exogenous nucleic acid may be introduced into the host to compensate only for those pathway enzymes that are not endogenously expressed in the host microorganism.

In an embodiment, the microorganism comprises a native butanoate pathway. For example, the microorganism may comprise one or more genes encoding enzymes and/or substrates necessary for the production or metabolism of butanoate (also known as butyrate). In an embodiment, the microorganism may endogenously express one or more of the following butanoate pathway enzymes: acetyl-CoA acetyltransferase (also known as thiolase), 3-hydroxybutyryl-CoA dehydrogenase, 3-hydroxybutyryl-CoA dehydratase (also known as crotonase), butyryl-CoA dehydrogenase, trans-2-enoyl-CoA reductase, CoA-transferase subunit A, CoA-transferase subunit B, acetaldehyde/alcohol dehydrogenase, butanol dehydrogenase, aldehyde:ferredwdn oxidoreductase, acetoacetate decarboxylase, and/or secondary alcohol dehydrogenase.

In an embodiment, the microorganism is genetically engineered to inhibit native production of butanoate and to thereby force increased expression of a bioproduct of interest such as crotyl alcohol. For example, crotonyl-CoA production in the microorganism host may be enhanced by disruption of butyryl-CoA dehydrogenase (BCD) expression of the butanoate pathway.

While a genomic deletion is a preferred embodiment for decreasing or silencing gene expression, any genomic mutation resulting in inactivation of the enzyme would be sufficient, including but not limited to partial gene deletion, nonsense mutation, transcriptional promoter deletion, etc. In another embodiment, the transcriptional expression of this gene can be reduced by using antisense RNA.

In an embodiment, the microorganism may be a bacteria or yeast or fungus capable of metabolizing CO₂. The organism may be autotrophic. In an embodiment, the organism may be capable of assimilating CO, CO₂, methanol, etc., for growth. The organism may also be capable of utilizing glycolysis for growth. In certain embodiments, the microorganism may be mixotrophic such that it is capable of assimilating CO, CO₂, methanol, etc., for growth and also capable of utilizing glycolysis for growth, either concurrently or at various stages of growth or fermentation. According to an embodiment, said organism is acetogenic. For example, said organism may be acetogenic Clostridia. Mixotrophic fermentation methods and microorganisms for use in such methods are described in detail in PCT International Application No. PCT/US2016/019760 as well as U.S. patent application Ser. No. 15/055,045.

In an embodiment, the microorganism may comprise a native butanoate metabolic pathway and have a genetic deletion of the BCD and/or TER genes. BCD and/or TER expression may alternatively be disrupted or silenced by other mechanisms. Examples of microorganism comprising a native butanoate metabolic pathway include Clostridium carboxidovorans, Eubacterium limosum, Butyribacterium methylotrophicum, Clostridium acetobutylicum, Clostridium beijerinckii, Clostridium kluyveri, Clostridium butyricum, Clostridium tyrobutyricum, Clostridium cellulovorans, Clostridium pasteurianum, Clostridium saccharoperbutylacetonicum, and Clostridium saccharobutylicum. In an embodiment, such a microorganism may be capable of redirecting crotonyl-CoA into crotyl alcohol. Such a microorganism may also be capable of producing acetone and/or isopropanol. Such a microorganism may be mixotrophic or non-mixotrophic.

In an embodiment, the microorganism may be mixotrophic and comprise a native butanoate metabolic pathway and have a genetic deletion of the BCD and/or TER genes. BCD and/or TER expression may alternatively be disrupted or silenced by other mechanisms. In an embodiment, such a microorganism may be capable of redirecting crotonyl-CoA into crotyl alcohol. Such a microorganism may also be capable of producing acetone and/or isopropanol.

### II. EXEMPLARY POLYNUCLEOTIDE AND AMINO ACIDS SEQUENCES OF THE INVENTION

An exemplary acetaldehyde dehydrogenase (ALDH) for use in the present invention catalyzes a CoA-acylating reaction in which crotonyl-CoA is converted into crotonaldehyde. Any similar substrates can also be used, such as acetyl-CoA into acetaldehyde, butyryl-CoA into butyraldehyde, and others. This reaction typically requires a coenzyme, such as NADH or NADPH. Exemplary nucleic acid and amino acid sequences are set forth below:

EC number: 1.2.1.10 or 1.2.1.57

Example nucleic acid sequence:

ATGAATAAAGACACACTAATACCTACAACTAAAGATTTAAAAGTAAAAAC
AAATGGTGAAAACATTAATTTAAAGAACTACAAGGATAATTCTTCATGTT
TCGGAGTATTCGAAAATGTTGAAAATGCTATAAGCAGCGCTGTACACGCA
CAAAAGATATTATCCCTTCATTATACAAAAGAGCAAAGAGAAAAAATCAT
AACTGAGATAAGAAAGGCCGCATTACAAAATAAAGAGGTCTTGGCTACAA
TGATTCTAGAAGAAACACATATGGGAAGATATGAGGATAAAATATTAAAA
CATGAATTGGTAGCTAAATATACTCCTGGTACAGAAGATTTAACTACTAC
TGCTTGGTCAGGTGATAATGGTCTTACAGTTGTAGAAATGTCTCCATATG
GTGTTATAGGTGCAATAACTCCTTCTACGAATCCAACTGAAACTGTAATA
TGTAATAGCATAGGCATGATAGCTGCTGGAAATGCTGTAGTATTTAACGG
ACACCCATGCGCTAAAAAATGTGTTGCCTTTGCTGTTGAAATGATAAATA
AGGCAATTATTTCATGTGGCGGTCCTGAAAATCTAGTAACAACTATAAAA
AATCCAACTATGGAGTCTCTAGATGCAATTATTAAGCATCCTTCAATAAA
ACTTCTTTGCGGAACTGGGGGTCCAGGAATGGTAAAAACCCTCTTAAATT
CTGGTAAGAAAGCTATAGGTGCTGGTGCTGGAAATCCACCAGTTATTGTA
GATGATACTGCTGATATAGAAAAGGCTGGTAGGAGCATCATTGAAGGCTG
TTCTTTTGATAATAATTTACCTTGTATTGCAGAAAAAGAAGTATTTGTTT
TTGAGAATGTTGCAGATGATTTAATATCTAACATGCTAAAAAATAATGCT
GTAATTATAAATGAAGATCAAGTATCAAAATTAATAGATTTAGTATTACA
AAAAAATAATGAAACTCAAGAATACTTTATAAACAAAAAATGGGTAGGAA
AAGATGCAAAATTATTCTTAGATGAAATAGATGTTGAGTCTCCTTCAAAT
GTTAAATGCATAATCTGCGAAGTAAATGCAAATCATCCATTTGTTATGAC
AGAACTCATGATGCCAATATTGCCAATTGTAAGAGTTAAAGATATAGATG
AAGCTATTAAATATGCAAAGATAGCAGAACAAAATAGAAAACATAGTGCC
TATATTTATTCTAAAAATATAGACAACCTAAATAGATTTGAAAGAGAAAT
AGATACTACTATTTTTGTAAAGAATGCTAAATCTTTTGCTGGTGTTGGTT
ATGAAGCAGAAGGATTTACAACTTTCACTATTGCTGGATCTACTGGTGAG
GGAATAACCTCTGCAAGGAATTTTACAAGACAAAGAAGATGTGTACTTGC
CGGCTAA

Example amino acid sequence:

MNKDTLIPTTKDLKVKINGENINLKNYKDNSSCFGVFENVENAISSAVHA
QKILSLHYTKEQREKIITEIRKAALQNKEVLATMILEETHMGRYEDKILK
HELVAKYTPGTEDLTTTAWSGDNGLTVVEMSPYGVIGAITPSTNPTETVI
CNSIGMIAAGNAVVFNGHPCAKKCVAFAVEMINKAIISCGGPENLVTTIK
NPTMESLDAIIKHPSIKLLCGTGGPGMVKTLLNSGKKAIGAGAGNPPVIV
DDTADIEKAGRSIIEGCSFDNNLPCIAEKEVFVFENVADDLISNMLKNNA
VIINEDQVSKLIDLVLQKNNETQEYFINKKWVGKDAKLFLDEIDVESPSN
VKCIICEVNANHPFVMTELMMPILPIVRVKDIDEAIKYAKIAEQNRKHSA
YIYSKNIDNLNRFEREIDTTIFVKNAKSFAGVGYEAEGFTTFTIAGSTGE
GITSARNFTRQRRCVLAG

An exemplary alcohol dehydrogenase (ADH) for use in the present invention catalyzes the dehydrogenation of an aldehyde into an alcohol, particularly crotonaldehyde into crotyl alcohol, though any aldehyde can be a substrate. This reaction typically requires a coenzyme, such as NADH or NADPH. This enzyme can also be known as a butanol dehydrogenase (BDH). Exemplary nucleic acid and amino acid sequences are set forth below:

EC number: 1.1.1.1

Example nucleic acid sequence:

GTGGTTGATTTCGAATATTCAATACCAACTAGAATTTTTTTCGGTAAAGA
TAAGATAAATGTACTTGGAAGAGAGCTTAAAAAATATGGTTCTAAAGTGC
TTATAGTTTATGGTGGAGGAAGTATAAAGAGAAATGGAATATATGATAAA
GCTGTAAGTATACTTGAAAAAAACAGTATTAAATTTTATGAACTTGCAGG
AGTAGAGCCAAATCCAAGAGTAACTACAGTTGAAAAAGGAGTTAAAATAT
GTAGAGAAAATGGAGTTGAAGTAGTACTAGCTATAGGTGGAGGAAGTGCA
ATAGATTGCGCAAAGGTTATAGCAGCAGCATGTGAATATGATGGAAATCC
ATGGGATATTGTGTTAGATGGCTCAAAAATAAAAAGGGTGCTTCCTATAG
CTAGTATATTAACCATTGCTGCAACAGGATCAGAAATGGATACGTGGGCA
GTAATAAATAATATGGATACAAACGAAAAACTAATTGCGGCACATCCAGA
TATGGCTCCTAAGTTTTCTATATTAGATCCAACGTATACGTATACCGTAC
CTACCAATCAAACAGCAGCAGGAACAGCTGATATTATGAGTCATATATTT
GAGGTGTATTTTAGTAATACAAAAACAGCATATTTGCAGGATAGAATGGC
AGAAGCGTTATTAAGAACTTGTATTAAATATGGAGGAATAGCTCTTGAGA
AGCCGGATGATTATGAGGCAAGAGCCAATCTAATGTGGGCTTCAAGTCTT
GCGATAAATGGACTTTTAACATATGGTAAAGACACTAATTGGAGTGTACA
CTTAATGGAACATGAATTAAGTGCTTATTACGACATAACACACGGCGTAG
GGCTTGCAATTTTAACACCTAATTGGATGGAGTATATTTTAAATAATGAT
ACAGTGTACAAGTTTGTTGAATATGGTGTAAATGTTTGGGGAATAGACAA
AGAAAAAAATCACTATGACATAGCACATCAAGCAATACAAAAAACAAGAG
ATTACTTTGTAAATGTACTAGGTTTACCATCTAGACTGAGAGATGTTGGA
ATTGAAGAAGAAAAATTGGACATAATGGCAAAGGAATCAGTAAAGCTTAC
AGGAGGAACCATAGGAAACCTAAGACCAGTAAACGCCTCCGAAGTCCTAC
AAATATTCAAAAAATCTGTGTAA

Example amino acid sequence:

MVDFEYSIPTRIFFGKDKINVLGRELKKYGSKVLIVYGGGSIKRNGIYDK
AVSILEKNSIKFYELAGVEPNPRVTTVEKGVKICRENGVEVVLAIGGGSA
IDCAKVIAAACEYDGNPWDIVLDGSKIKRVLPIASILTIAATGSEMDTWA
VINNMDTNEKLIAAHPDMAPKFSILDPTYTYTVPTNQTAAGTADIMSHIF
EVYFSNIKTAYLQDRMAEALLRICIKYGGIALEKPDDYEARANLMWASSL
AINGLLTYGKDTNWSVHLMEHELSAYYDITHGVGLAILTPNWMEYILNND
TVYKFVEYGVNVWGIDKEKNHYDIAHQAIQKTRDYFVNVLGLPSRLRDVG
IEEEKLDIMAKESVKLTGGTIGNLRPVNASEVLQIFKKSV

An exemplary bifunctional acetaldehyde/alcohol dehydrogenase (ADHE) for use in the present invention is a bifunctional enzyme that catalyzes two reactions sequentially. The first reaction is a CoA-acylating reaction in which crotonyl-CoA is converted into crotonaldehyde. The second reaction is a dehydrogenase reaction in which crotonaldehyde is converted into crotyl alcohol. Any similar substrates can also be used, such as acetyl-CoA, butyryl-CoA, and others. This reaction typically requires a coenzyme, such as NADH or NADPH. Exemplary nucleic acid and amino acid sequences are set forth below:

EC number: For the first reaction (1.2.1.10 or 1.2.1.57); for the second reaction (1.1.1.1)

Example nucleic acid sequence:

ATGAAAGTCACAACAGTAAAGGAATTAGATGAAAAACTCAAGGTAATTAA
AGAAGCTCAAAAAAAATTCTCTTGTTACTCGCAAGAAATGGTTGATGAAA
TCTTTAGAAATGCAGCAATGGCAGCAATCGACGCAAGGATAGAGCTAGCA
AAAGCAGCTGTTTTGGAAACCGGTATGGGCTTAGTTGAAGACAAGGTTAT
AAAAAATCATTTTGCAGGCGAATACATCTATAACAAATATAAGGATGAAA
AAACCTGCGGTATAATTGAACGAAATGAACCCTACGGAATTACAAAAATA
GCAGAACCTATAGGAGTTGTAGCTGCTATAATCCCTGTAACAAACCCCAC
ATCAACAACAATATTTAAATCCTTAATATCCCTTAAAACTAGAAATGGAA
TTTTCTTTTCGCCTCACCCAAGGGCAAAAAAATCCACAATACTAGCAGCT
AAAACAATACTTGATGCAGCCGTTAAGAGTGGTGCCCCGGAAAATATAAT
AGGTTGGATAGATGAACCTTCAATTGAACTAACTCAATATTTAATGCAAA
AAGCAGATATAACCCTTGCAACTGGTGGTCCCTCACTAGTTAAATCTGCT
TATTCTTCCGGAAAACCAGCAATAGGTGTTGGTCCGGGTAACACCCCAGT
AATAATTGATGAATCTGCTCATATAAAAATGGCAGTAAGTTCAATTATAT
TATCCAAAACCTATGATAATGGTGTTATATGTGCTTCTGAACAATCTGTA
ATAGTCTTAAAATCCATATATAACAAGGTAAAAGATGAGTTCCAAGAAAG
AGGAGCTTATATAATAAAGAAAAACGAATTGGATAAAGTCCGTGAAGTGA
TTTTTAAAGATGGATCCGTAAACCCTAAAATAGTCGGACAGTCAGCTTAT
ACTATAGCAGCTATGGCTGGCATAAAAGTACCTAAAACCACAAGAATATT
AATAGGAGAAGTTACCTCCTTAGGTGAAGAAGAACCTTTTGCCCACGAAA
AACTATCTCCTGTTTTGGCTATGTATGAGGCTGACAATTTTGATGATGCT
TTAAAAAAAGCAGTAACTCTAATAAACTTAGGAGGCCTCGGCCATACCTC
AGGAATATATGCAGATGAAATAAAAGCACGAGATAAAATAGATAGATTTA
GTAGTGCCATGAAAACCGTAAGAACCTTTGTAAATATCCCAACCTCACAA
GGTGCAAGTGGAGATCTATATAATTTTAGAATACCACCTTCTTTCACGCT
TGGCTGCGGATTTTGGGGAGGAAATTCTGTTTCCGAGAATGTTGGTCCAA
AACATCTTTTGAATATTAAAACCGTAGCTGAAAGGAGAGAAAACATGCTT
TGGTTTAGAGTTCCACATAAAGTATATTTTAAGTTCGGTTGTCTTCAATT
TGCTTTAAAAGATTTAAAAGATCTAAAGAAAAAAAGAGCCTTTATAGTTA
CTGATAGTGACCCCTATAATTTAAACTATGTTGATTCAATAATAAAAATA
CTTGAGCACCTAGATATTGATTTTAAAGTATTTAATAAGGTTGGAAGAGA
AGCTGATCTTAAAACCATAAAAAAAGCAACTGAAGAAATGTCCTCCTTTA
TGCCAGACACTATAATAGCTTTAGGTGGTACCCCTGAAATGAGCTCTGCA
AAGCTAATGTGGGTACTATATGAACATCCAGAAGTAAAATTTGAAGATCT
TGCAATAAAATTTATGGACATAAGAAAGAGAATATATACTTTCCCAAAAC
TCGGTAAAAAGGCTATGTTAGTTGCAATTACAACTTCTGCTGGTTCCGGT
TCTGAGGTTACTCCTTTTGCTTTAGTAACTGACAATAACACTGGAAATAA
GTACATGTTAGCAGATTATGAAATGACACCAAATATGGCAATTGTAGATG
CAGAACTTATGATGAAAATGCCAAAGGGATTAACCGCTTATTCAGGTATA
GATGCACTAGTAAATAGTATAGAAGCATACACATCCGTATATGCTTCAGA
ATACACAAACGGACTAGCACTAGAGGCAATACGATTAATATTTAAATATT
TGCCTGAGGCTTACAAAAACGGAAGAACCAATGAAAAAGCAAGAGAGAAA
ATGGCTCACGCTTCAACTATGGCAGGTATGGCATCCGCTAATGCATTTCT
AGGTCTATGTCATTCCATGGCAATAAAATTAAGTTCAGAACACAATATTC
CTAGTGGCATTGCCAATGCATTACTAATAGAAGAAGTAATAAAATTTAAC
GCAGTTGATAATCCTGTAAAACAAGCCCCTTGCCCACAATATAAGTATCC
AAACACCATATTTAGATATGCTCGAATTGCAGATTATATAAAGCTTGGAG
GAAATACTGATGAGGAAAAGGTAGATCTCTTAATTAACAAAATACATGAA
CTAAAAAAAGCTTTAAATATACCAACTTCAATAAAGGATGCAGGTGTTTT
GGAGGAAAACTTCTATTCCTCCCTTGATAGAATATCTGAACTTGCACTAG
ATGATCAATGCACAGGCGCTAATCCTAGATTTCCTCTTACAAGTGAGATA
AAAGAAATGTATATAAATTGTTTTAAAAAACAACCTTAA

Example amino acid sequence:

MKVTTVKELDEKLKVIKEAQKKFSCYSQEMVDEIFRNAAMAAIDARIELA
KAAVLETGMGLVEDKVIKNHFAGEYIYNKYKDEKTCGIIERNEPYGITKI
AEPIGVVAAIIPVTNPTSTTIFKSLISLKTRNGIFFSPHPRAKKSTILAA
KTILDAAVKSGAPENIIGWIDEPSIELTQYLMQKADITLATGGPSLVKSA
YSSGKPAIGVGPGNTPVIIDESAHIKMAVSSIILSKTYDNGVICASEQSV
IVLKSIYNKVKDEFQERGAYIIKKNELDKVREVIFKDGSVNPKIVGQSAY
TIAAMAGIKVPKTTRILIGEVTSLGEEEPFAHEKLSPVLAMYEADNFDDA
LKKAVTLINLGGLGHTSGIYADEIKARDKIDRFSSAMKTVRTFVNIPTSQ
GASGDLYNFRIPPSFTLGCGFWGGNSVSENVGPKHLLNIKTVAERRENML
WERVPHKVYFKFGCLQFALKDLKDLKKKRAFIVTDSDPYNLNYVDSIIKI
LEHLDIDEKVENKVGREADLKTIKKATEEMSSFMPDTIIALGGTPEMSSA
KLMWVLYEHPEVKFEDLAIKFMDIRKRIYTFPKLGKKAMLVAITTSAGSG
SEVTPFALVTDNNTGNKYMLADYEMTPNMAIVDAELMMKMPKGLTAYSGI
DALVNSIEAYTSVYASEYTNGLALEAIRLIFKYLPEAYKNGRTNEKAREK
MAHASTMAGMASANAFLGLCHSMAIKLSSEHNIPSGIANALLIEEVIKFN
AVDNPVKQAPCPQYKYPNTIFRYARIADYIKLGGNTDEEKVDLLINKIHE
LKKALNIPTSIKDAGVLEENFYSSLDRISELALDDQCTGANPRFPLTSEI
KEMYINCFKKQP

An exemplary aldehyde oxidoreductase (AOR), also known as an aldehyde:ferredoxin oxidoreductase, for use in the present invention catalyzes the reduction of a carboxylic acid into its corresponding aldehyde. For example, crotonic acid into crotonaldehyde. This reaction typically requires a coenzyme, such as ferredoxin. Exemplary nucleic acid and amino acid sequences are set forth below:

EC number: 1.2.7.5

Example nucleic acid sequence:

ATGTACGGATATAAGGGTAAGGTATTAAGAATTAATCTAAGTAGTAAAAC
TTATATAGTGGAAGAATTGAAAATTGACAAAGCTAAAAAATTTATAGGTG
CAAGAGGGTTAGGCGTAAAAACCTTATTTGACGAAGTAGATCCAAAGGTA
GATCCATTATCACCTGATAACAAATTTATTATAGCAGCGGGACCACTTAC
AGGTGCACCTGTTCCAACAAGCGGAAGATTCATGGTAGTTACTAAATCAC
CTTTAACAGGAACTATTGCTATTGCAAATTCAGGTGGAAAATGGGGAGCA
GAATTCAAAGCAGCTGGATACGATATGATAATCGTTGAAGGTAAATCTGA
TAAAGAAGTTTATGTAAATATAGTAGATGATAAAGTAGAATTTAGGGATG
CTTCTCATGTTTGGGGAAAACTAACAGAAGAAACTACAAAAATGCTTCAA
CAGGAAACAGATTCGAGAGCTAAGGTTTTATGCATAGGACCAGCTGGGGA
AAAGTTATCACTTATGGCAGCAGTTATGAATGATGTTGATAGAACAGCAG
GACGTGGTGGTGTTGGAGCTGTTATGGGTTCAAAGAACTTAAAAGCTATT
GTAGTTAAAGGAAGCGGAAAAGTAAAATTATTTGATGAACAAAAAGTGAA
GGAAGTAGCACTTGAGAAAACAAATATTTTAAGAAAAGATCCAGTAGCTG
GTGGAGGACTTCCAACATACGGAACAGCTGTACTTGTTAATATTATAAAT
GAAAATGGTGTACATCCAGTAAAGAATTTTCAAAAATCTTATACAGATCA
AGCAGATAAGATCAGTGGAGAAACTTTAACTAAAGATTGCTTAGTTAGAA
AAAATCCTTGCTATAGGTGTCCAATTGCCTGTGGAAGATGGGTAAAACTT
GATGATGGAACTGAATGTGGAGGACCAGAATATGAAACATTATGGTCATT
TGGATCTGATTGTGATGTATACGATATAAATGCTGTAAATACAGCAAATA
TGTTGTGTAATGAATATGGATTAGATACCATTACAGCAGGATGTACTATT
GCAGCAGCTATGGAACTTTATCAAAGAGGTTATATTAAGGATGAAGAAAT
AGCAGCAGATGGATTGTCACTTAATTGGGGAGATGCTAAGTCCATGGTTG
AATGGGTAAAGAAAATGGGACTTAGAGAAGGATTTGGAGACAAGATGGCA
GATGGTTCATACAGACTTTGTGACTCATACGGTGTACCTGAGTATTCAAT
GACTGTAAAAAAACAGGAACTTCCAGCATATGACCCAAGAGGAATACAGG
GACATGGTATTACTTATGCTGTTAACAATAGGGGAGGATGTCACATTAAG
GGATATATGGTAAGTCCTGAAATACTTGGCTATCCAGAAAAACTTGATAG
ACTTGCAGTGGAAGGAAAAGCAGGATATGCTAGAGTATTCCATGATTTAA
CAGCTGTTATAGATTCACTTGGATTATGTATTTTTACAACATTTGGTCTT
GGTGCACAGGATTATGTTGATATGTATAATGCAGTAGTTGGTGGAGAATT
ACATGATGTAAATTCTTTAATGTTAGCTGGAGATAGAATATGGACTTTAG
AAAAAATATTTAACTTAAAGGCAGGCATAGATAGTTCACAGGATACTCTT
CCAAAGAGATTGCTTGAAGAACAAATTCCAGAAGGACCATCAAAAGGAGA
AGTTCATAAGTTAGATGTACTACTACCTGAATATTATTCAGTACGTGGAT
GGGATAAAAATGGTATTCCTACAGAGGAAACGTTAAAGAAATTAGGATTA
GATGAATACGTAGGTAAGCTTTAG

Example amino acid sequence:

MYGYKGKVLRINLSSKTYIVEELKIDKAKKFIGARGLGVKTLFDEVDPKV
DPLSPDNKFIIAAGPLTGAPVPTSGREMVVIKSPLIGTIAIANSGGKWGA
EFKAAGYDMIIVEGKSDKEVYVNIVDDKVEFRDASHVWGKLTEETTKMLQ
QETDSRAKVLCIGPAGEKLSLMAAVMNDVDRTAGRGGVGAVMGSKNLKAI
VVKGSGKVKLFDEQKVKEVALEKTNILRKDPVAGGGLPTYGTAVLVNIIN
ENGVHPVKNFQKSYTDQADKISGETLTKDCLVRKNPCYRCPIACGRWVKL
DDGTECGGPEYETLWSFGSDCDVYDINAVNTANMLCNEYGLDTITAGCTI
AAAMELYQRGYIKDEEIAADGLSLNWGDAKSMVEWVKKMGLREGFGDKMA
DGSYRLCDSYGVPEYSMTVKKQELPAYDPRGIQGHGITYAVNNRGGCHIK
GYMVSPEILGYPEKLDRLAVEGKAGYARVFHDLTAVIDSLGLCIFTTFGL
GAQDYVDMYNAVVGGELHDVNSLMLAGDRIWTLEKIFNLKAGIDSSQDTL
PKRLLEEQIPEGPSKGEVHKLDVLLPEYYSVRGWDKNGIPTEETLKKLGL
DEYVGKL

An exemplary phosphotransacetylase (PTA) for use in the present invention catalyzes the conversion of crotonyl-CoA into crotonyl phosphate. This reaction requires a phosphate group to transfer onto the crotonyl substrate and releases a CoA group. Exemplary nucleic acid and amino acid sequences are set forth below:

EC number: 2.3.1.19

Example nucleic acid sequence:

GTGATTAAGAGTTTTAATGAAATTATCATGAAGGTAAAGAGCAAAGAAAT
GAAAAAAGTTGCTGTTGCTGTAGCACAAGACGAGCCAGTACTTGAAGCAG
TAAGAGATGCTAAGAAAAATGGTATTGCAGATGCTATTCTTGTTGGAGAC
CATGACGAAATCGTGTCAATCGCGCTTAAAATAGGAATGGATGTAAATGA
TTTTGAAATAGTAAACGAGCCTAACGTTAAGAAAGCTGCTTTAAAGGCAG
TAGAGCTTGTATCAACTGGAAAAGCTGATATGGTAATGAAGGGACTTGTA
AATACAGCAACTTTCTTAAGATCTGTATTAAACAAAGAAGTTGGACTTAG
AACAGGAAAAACTATGTCTCACGTTGCAGTATTTGAAACTGAGAAATTTG
ATAGACTATTATTTTTAACAGATGTTGCTTTCAATACTTATCCTGAATTA
AAGGAAAAAATTGATATAGTAAACAATTCAGTTAAGGTTGCACATGCAAT
AGGAATTGAAAATCCAAAGGTTGCTCCAATTTGTGCAGTTGAGGTTATAA
ACCCTAAAATGCCATCAACACTTGATGCAGCAATGCTTTCAAAAATGAGT
GACAGAGGACAAATTAAAGGTTGTGTAGTTGACGGACCTTTAGCACTTGA
TATAGCTTTATCAGAAGAAGCAGCACATCATAAGGGAGTAACAGGAGAAG
TTGCTGGAAAAGCTGATATCTTCTTAATGCCAAACATAGAAACAGGAAAT
GTAATGTATAAGACTTTAACATATACAACTGATTCAAAAAATGGAGGAAT
CTTAGTTGGAACTTCTGCACCAGTTGTTTTAACTTCAAGAGCTGACAGCC
ATGAAACAAAAATGAACTCTATAGCACTTGCAGCTTTAGTTGCAGGCAAT
AAATAA

Example amino acid sequence:

MIKSFNEIIMKVKSKEMKKVAVAVAQDEPVLEAVRDAKKNGIADAILVG
DHDEIVSIALKIGMDVNEFEIVNEPNVKKAALKAVELVSTGKADMVMKG
LVNTATFLRSVLNKEVGLRIGKTMSHVAVFETEKFDRLLFLTDVAENTY
PELKEKIDIVNNSVKVAHAIGIENPKVAPICAVEVINPKMPSTLDAAML
SKMSDRGQIKGCVVDGPLALDIALSEEAAHHKGVTGEVAGKADIFLMPN
IETGNVMYKTLTYTTDSKNGGILVGTSAPVVLTSRADSHETKMNSIALA
ALVAGNK

An exemplary acetate kinase (ACK) for use in the present invention catalyzes the conversion of crotonyl phosphate into crotonate while simultaneously generating a molecule of ATP. This reaction requires an ADP (adenosine diphosphate) onto which the phosphate from crotonyl phosphate is transferred to in order to generate the ATP (adenosine triphosphate). Exemplary nucleic acid and amino acid sequences are set forth below:

EC number: 2.7.2.7

Example nucleic acid sequence:

ATGTATAGATTACTAATAATCAATCCTGGCTCGACCTCAACTAAAATTGG
TATTTATGACGATGAAAAAGAGATATTTGAGAAGACTTTAAGACATTCAG
CTGAAGAGATAGAAAAATATAACACTATATTTGATCAATTTCAATTCAGA
AAGAATGTAATTTTAGATGCGTTAAAAGAAGCAAACATAGAAGTAAGTTC
TTTAAATGCTGTAGTTGGAAGAGGCGGACTCTTAAAGCCAATAGTAAGTG
GAACTTATGCAGTAAATCAAAAAATGCTTGAAGACCTTAAAGTAGGAGTT
CAAGGTCAGCATGCGTCAAATCTTGGTGGAATTATTGCAAATGAAATAGC
AAAAGAAATAAATGTTCCAGCATACATAGTTGATCCAGTTGTTGTGGATG
AGCTTGATGAAGTTTCAAGAATATCAGGAATGGCTGACATTCCAAGAAAA
AGTATATTCCATGCATTAAATCAAAAAGCAGTTGCTAGAAGATATGCAAA
AGAAGTTGGAAAAAAATACGAAGATCTTAATTTAATCGTAGTCCACATGG
GTGGAGGTACTTCAGTAGGTACTCATAAAGATGGTAGAGTAATAGAAGTT
AATAATACACTTGATGGAGAAGGTCCATTCTCACCAGAAAGAAGTGGTGG
AGTTCCAATAGGAGATCTTGTAAGATTGTGCTTCAGCAACAAATATACTT
ATGAAGAAGTAATGAAAAAGATAAACGGCAAAGGCGGAGTTGTTAGTTAC
TTAAATACTATCGATTTTAAGGCTGTAGTTGATAAAGCTCTTGAAGGAGA
TAAGAAATGTGCACTTATATATGAAGCTTTCACATTCCAGGTAGCAAAAG
AGATAGGAAAATGTTCAACCGTTTTAAAAGGAAATGTAGATGCAATAATC
TTAACAGGCGGAATTGCGTACAACGAGCATGTATGTAATGCCATAGAGGA
TAGAGTAAAATTCATAGCACCTGTAGTTAGATATGGTGGAGAAGATGAAC
TTCTTGCACTTGCAGAAGGTGGACTTAGAGTTTTAAGAGGAGAAGAAAAA
GCTAAGGAATACAAATAA

Example amino acid sequence:

MYRLLIINPGSTSTKIGIYDDEKEIFEKTLRHSAEEIEKYNTIFDQFQFR
KNVILDALKEANIEVSSLNAVVGRGGLLKPIVSGTYAVNQKMLEDLKVGV
QGQHASNLGGIIANEIAKEINVPAYIVDPVVVDELDEVSRISGMADIPRK
SIFHALNQKAVARRYAKEVGKKYEDLNLIVVHMGGGTSVGTHKDGRVIEV
NNTLDGEGPFSPERSGGVPIGDLVRLCFSNKYTYEEVMKKINGKGGVVSY
LNTIDFKAVVDKALEGDKKCALIYEAFTFQVAKEIGKCSTVLKGNVDAII
LTGGIAYNEHVCNAIEDRVKFIAPVVRYGGEDELLALAEGGLRVLRGEEK
AKEYK

An exemplary CoA-transferase subunit A (COAT-A) for use in the present invention catalyzes the transfer of coenzyme-A (CoA) between two molecules. For example, from acetoacetyl-CoA to acetate to form acetoacetate and acetyl-CoA or from acetoacetyl-CoA to crotonate to form acetoacetate and crotonyl-CoA. Exemplary subunit A nucleic acid and amino acid sequences are set forth below:

EC number: 2.8.3.8 or 2.8.3.9 or other related enzymes

Example nucleic acid sequence:

ATGAACTCTAAAATAATTAGATTTGAAAATTTAAGGTCATTCTTTAAAGA
TGGGATGACAATTATGATTGGAGGTTTTTTAAACTGTGGCACTCCAACCA
AATTAATTGATTTTTTAGTTAATTTAAATATAAAGAATTTAACGATTATA
AGTAATGATACATGTTATCCTAATACAGGTATTGGTAAGTTAATATCAAA
TAATCAAGTAAAAAAGCTTATTGCTTCATATATAGGCAGCAACCCAGATA
CTGGCAAAAAACTTTTTAATAATGAACTTGAAGTAGAGCTCTCTCCCCAA
GGAACTCTAGTGGAAAGAATACGTGCAGGCGGATCTGGCTTAGGTGGTGT
ACTAACTAAAACAGGTTTAGGAACTTTGATTGAAAAAGGAAAGAAAAAAA
TATCTATAAATGGAACGGAATATTTGTTAGAGCTACCTCTTACAGCCGAT
GTAGCATTAATTAAAGGTAGTATTGTAGATGAGGCCGGAAACACCTTCTA
TAAAGGTACTACTAAAAACTTTAATCCCTATATGGCAATGGCAGCTAAAA
CCGTAATAGTTGAAGCTGAAAATTTAGTTAGCTGTGAAAAACTAGAAAAG
GAAAAAGCAATGACCCCCGGAGTTCTTATAAATTATATAGTAAAGGAGCC
TGCATAA

Example amino acid sequence:

MNSKIIRFENLRSFFKDGMTIMIGGFLNCGTPTKLIDFLVNLNIKNLTII
SNDTCYPNTGIGKLISNNQVKKLIASYIGSNPDTGKKLFNNELEVELSPQ
GTLVERIRAGGSGLGGVLTKTGLGTLIEKGKKKISINGTEYLLELPLTAD
VALIKGSIVDEAGNTFYKGTTKNENPYMAMAAKTVIVEAENLVSCEKLEK
EKAMTPGVLINYIVKEPA

An exemplary CoA-transferase subunit B (COAT-B) for use in the present invention catalyzes the transfer of coenzyme-A (CoA) between two molecules. For example, from acetoacetyl-CoA to acetate to form acetoacetate and acetyl-CoA or from acetoacetyl-CoA to crotonate to form acetoacetate and crotonyl-CoA. Exemplary subunit B nucleic acid and amino acid sequences are set forth below:

EC number: 2.8.3.8 or 2.8.3.9 or other related enzymes

Example nucleic acid sequence:

ATGATTAATGATAAAAACCTAGCGAAAGAAATAATAGCCAAAAGAGTTGC
AAGAGAATTAAAAAATGGTCAACTTGTAAACTTAGGTGTAGGTCTTCCTA
CCATGGTTGCAGATTATATACCAAAAAATTTCAAAATTACTTTCCAATCA
GAAAACGGAATAGTTGGAATGGGCGCTAGTCCTAAAATAAATGAGGCAGA
TAAAGATGTAGTAAATGCAGGAGGAGACTATACAACAGTACTTCCTGACG
GCACATTTTTCGATAGCTCAGTTTCGTTTTCACTAATCCGTGGTGGTCAC
GTAGATGTTACTGTTTTAGGGGCTCTCCAGGTAGATGAAAAGGGTAATAT
AGCCAATTGGATTGTTCCTGGAAAAATGCTCTCTGGTATGGGTGGAGCTA
TGGATTTAGTAAATGGAGCTAAGAAAGTAATAATTGCAATGAGACATACA
AATAAAGGTCAACCTAAAATTTTAAAAAAATGTACACTTCCCCTCACGGC
AAAGTCTCAAGCAAATCTAATTGTAACAGAACTTGGAGTAATTGAGGTTA
TTAATGATGGTTTACTTCTCACTGAAATTAATAAAAACACAACCATTGAT
GAAATAAGGTCTTTAACTGCTGCAGATTTACTCATATCCAATGAACTTAG
ACCCATGGCTGTTTAG

Example amino acid sequence:

MINDKNLAKEIIAKRVARELKNGQLVNLGVGLPTMVADYIPKNFKITFQS
ENGIVGMGASPKINEADKDVVNAGGDYTTVLPDGTFFDSSVSFSLIRGGH
VDVTVLGALQVDEKGNIANWIVPGKMLSGMGGAMDLVNGAKKVIIAMRHT
NKGQPKILKKCTLPLTAKSQANLIVTELGVIEVINDGLLLTEINKNTTID
EIRSLTAADLLISNELRPMAV

An exemplary acetoacetate decarboxylase (ADC) for use in the present invention catalyzes the decarboxylation of acetoacetate into acetone and CO₂. Exemplary nucleic acid and amino acid sequences are set forth below:

EC number: 4.1.1.4

Example nucleic acid sequence:

ATGTTAAAGGATGAAGTAATTAAACAAATTAGCACGCCATTAACTTCGCC
TGCATTTCCTAGAGGACCCTATAAATTTCATAATCGTGAGTATTTTAACA
TTGTATATCGTACAGATATGGATGCACTTCGTAAAGTTGTGCCAGAGCCT
TTAGAAATTGATGAGCCCTTAGTCAGGTTTGAAATTATGGCAATGCATGA
TACGAGTGGACTTGGTTGTTATACAGAAAGCGGACAGGCTATTCCCGTAA
GCTTTAATGGAGTTAAGGGAGATTATCTTCATATGATGTATTTAGATAAT
GAGCCTGCAATTGCAGTAGGAAGGGAATTAAGTGCATATCCTAAAAAGCT
CGGGTATCCAAAGCTTTTTGTGGATTCAGATACTTTAGTAGGAACTTTAG
ACTATGGAAAACTTAGAGTTGCGACAGCTACAATGGGGTACAAACATAAA
GCCTTAGATGCTAATGAAGCAAAGGATCAAATTTGTCGCCCTAATTATAT
GTTGAAAATAATACCCAATTATGATGGAAGCCCTAGAATATGTGAGCTTA
TAAATGCGAAAATCACAGATGTTACCGTACATGAAGCTTGGACAGGACCA
ACTCGACTGCAGTTATTTGATCACGCTATGGCGCCACTTAATGATTTGCC
AGTAAAAGAGATTGTTTCTAGCTCTCACATTCTTGCAGATATAATATTGC
CTAGAGCTGAAGTTATATATGATTATCTTAAGTAA

Example amino acid sequence:

MLKDEVIKQISTPLTSPAFPRGPYKEHNREYFNIVYRTDMDALRKVVPEP
LEIDEPLVRFEIMAMHDTSGLGCYTESGQAIPVSFNGVKGDYLHMMYLDN
EPAIAVGRELSAYPKKLGYPKLFVDSDTLVGTLDYGKLRVATATMGYKHK
ALDANEAKDQICRPNYMLKIIPNYDGSPRICELINAKITDVTVHEAWTGP
TRLQLFDHAMAPLNDLPVKEIVSSSHILADIILPRAEVIYDYLK

An exemplary secondary alcohol dehydrogenase (SADH) for use in the present invention catalyzes the reduction of a ketone into a secondary alcohol. For example, acetone into 2-propanol (a.k.a. isopropanol). Exemplary nucleic acid and amino acid sequences are set forth below:

EC number: 1.1.1.1

Example nucleic acid sequence:

ATGAAAGGTTTTGCAATGTTAGGTATTAACAAATTAGGATGGATTGAAAA
GAAAAACCCAGTGCCAGGTCCTTATGATGCGATTGTACATCCTCTAGCTG
TATCCCCATGTACATCAGATATACATACGGTTTTTGAAGGAGCACTTGGT
AATAGGGAAAATATGATTTTAGGCCATGAAGCTGTAGGTGAAATAGCCGA
AGTTGGCAGCGAAGTTAAAGATTTTAAAGTTGGCGATAGAGTTATCGTAC
CATGCACAACACCTGACTGGAGATCTTTAGAAGTCCAAGCTGGTTTTCAG
CAGCATTCAAACGGTATGCTTGCAGGATGGAAGTTTTCCAATTTTAAAGA
TGGTGTATTTGCAGATTACTTTCATGTAAACGATGCAGATATGAATCTTG
CCATACTCCCAGATGAAATACCTTTAGAAAGTGCAGTTATGATGACAGAC
ATGATGACTACTGGTTTTCATGGAGCAGAACTTGCAGACATAAAAATGGG
CTCCAGCGTTGTAGTAATTGGTATAGGAGCTGTTGGATTAATGGGAATAG
CCGGTTCCAAACTTCGAGGAGCAGGCAGAATTATCGGTGTTGGAAGCAGA
CCTGTTTGTGTTGAAACAGCTAAATTTTATGGAGCAACTGATATTGTAAA
TTATAAAAATGGTGATATAGTTGAACAAATCATGGACTTAACTCATGGTA
AAGGTGTAGACCGTGTAATCATGGCAGGCGGTGGTGCTGAAACACTAGCA
CAAGCAGTAACTATGGTTAAACCTGGCGGCGTAATTTCTAACATCAACTA
CCATGGAAGCGGTGATACTTTACCAATACCTCGTGTTCAATGGGGCTGCG
GCATGGCTCACAAAACTATAAGAGGAGGATTATGCCCCGGCGGACGTCTT
AGAATGGAAATGCTAAGAGATCTTGTTCTATATAAACGTGTTGATTTGAG
TAAACTTGTTACTCATGTATTTGATGGTGCAGAAAATATTGAAAAGGCCC
TTTTGCTTATGAAAAATAAGCCAAAAGATTTAATTAAATCAGTAGTTACA
TTCTAA

Example amino acid sequence:

MKGFAMLGINKLGWIEKKNPVPGPYDAIVHPLAVSPCTSDIHTVFEGAL
GNRENMILGHEAVGEIAEVGSEVKDFKVGDRVIVPCTTPDWRSLEVQAG
FQQHSNGMLAGWKESNEKDGVFADYFHVNDADMNLAILPDEIPLESAVM
MTDMMTTGFHGAELADIKMGSSVVVIGIGAVGLMGIAGSKLRGAGRIIG
VGSRPVCVETAKFYGATDIVNYKNGDIVEQIMDLTHGKGVDRVIMAGGG
AETLAQAVTMVKPGGVISNINYHGSGDTLPIPRVQWGCGMAHKTIRGGL
CPGGRLRMEMLRDLVLYKRVDLSKLVTHVEDGAENIEKALLLMKNKPKD
LIKSVVTF

An exemplary butyryl-CoA dehydrogenase (BCD) for use in the present invention catalyzes the reduction of crotonyl-CoA into butyryl-CoA by reducing the carbon-carbon double bond in crotonyl-CoA. This enzyme requires an electron-transfer flavoprotein. Exemplary nucleic acid and amino acid sequences are set forth below:

EC number: 1.3.8.1

Example nucleic acid sequence:

ATGGATTTTAATTTAACAAGAGAACAAGAATTAGTAAGACAGATGGTTAG
AGAATTTGCTGAAAATGAAGTTAAACCTATAGCAGCAGAAATTGATGAAA
CAGAAAGATTTCCAATGGAAAATGTAAAGAAAATGGGTCAGTATGGTATG
ATGGGAATTCCATTTTCAAAAGAGTATGGTGGCGCAGGTGGAGATGTATT
ATCTTATATAATCGCCGTTGAGGAATTATCAAAGGTTTGCGGTACTACAG
GAGTTATTCTTTCAGCACATACATCACTTTGTGCTTCATTAATAAATGAA
CATGGTACAGAAGAACAAAAACAAAAATATTTAGTACCTTTAGCTAAAGG
TGAAAAAATAGGTGCTTATGGATTGACTGAGCCAAATGCAGGAACAGATT
CTGGAGCACAACAAACAGTAGCTGTACTTGAAGGAGATCATTATGTAATT
AATGGTTCAAAAATATTCATAACTAATGGAGGAGTTGCAGATACTTTTGT
TATATTTGCAATGACTGACAGAACTAAAGGAACAAAAGGTATATCAGCAT
TTATAATAGAAAAAGGCTTCAAAGGTTTCTCTATTGGTAAAGTTGAACAA
AAGCTTGGAATAAGAGCTTCATCAACAACTGAACTTGTATTTGAAGATAT
GATAGTACCAGTAGAAAACATGATTGGTAAAGAAGGAAAAGGCTTCCCTA
TAGCAATGAAAACTCTTGATGGAGGAAGAATTGGTATAGCAGCTCAAGCT
TTAGGTATAGCTGAAGGTGCTTTCAACGAAGCAAGAGCTTACATGAAGGA
GAGAAAACAATTTGGAAGAAGCCTTGACAAATTCCAAGGTCTTGCATGGA
TGATGGCAGATATGGATGTAGCTATAGAATCAGCTAGATATTTAGTATAT
AAAGCAGCATATCTTAAACAAGCAGGACTTCCATACACAGTTGATGCTGC
AAGAGCTAAGCTTCATGCTGCAAATGTAGCAATGGATGTAACAACTAAGG
CAGTACAATTATTTGGTGGATACGGATATACAAAAGATTATCCAGTTGAA
AGAATGATGAGAGATGCTAAGATAACTGAAATATATGAAGGAACTTCAGA
AGTTCAGAAATTAGTTATTTCAGGAAAAATTTTTAGATAA

Example amino acid sequence:

MDFNLTREQELVRQMVREFAENEVKPIAAEIDETERFPMENVKKMGQYGM
MGIPFSKEYGGAGGDVLSYIIAVEELSKVCGTTGVILSAHTSLCASLINE
HGTEEQKQKYLVPLAKGEKIGAYGLTEPNAGTDSGAQQTVAVLEGDHYVI
NGSKIFITNGGVADTEVIFAMTDRTKGTKGISAFIIEKGFKGESIGKVEQ
KLGIRASSTTELVFEDMIVPVENMIGKEGKGFPIAMKTLEGGRIGIAAQA
LGIAEGAFNEARAYMKERKQFGRSLDKFQGLAWMMADMDVAIESARYLVY
KAAYLKQAGLPYTVDAARAKLHAANVAMDVITKAVQLEGGYGYTKEYPVE
RMMRDAKITEIYEGTSEVQKLVISGKIFR

An exemplary trans-2-enoyl-CoA reductase (TER) for use in the present invention catalyzes the reduction of crotonyl-CoA into butyryl-CoA by reducing the carbon-carbon double bond in crotonyl-CoA. Exemplary nucleic acid and amino acid sequences are set forth below:

EC number: 1.3.1.44

Example nucleic acid sequence:

ATGATAGTAAAAGCAAAGTTTGTAAAAGGATTTATCAGAGATGTACATCC
TTATGGTTGCAGAAGGGAAGTACTAAATCAAATAGATTATTGTAAGAAGG
CTATTGGGTTTAGGGGACCAAAGAAGGTTTTAATTGTTGGAGCCTCATCT
GGGTTTGGTCTTGCTACTAGAATTTCAGTTGCATTTGGAGGTCCAGAAGC
TCACACAATTGGAGTATCCTATGAAACAGGAGCTACAGATAGAAGAATAG
GAACAGCGGGATGGTATAATAACATATTTTTTAAAGAATTTGCTAAAAAA
AAAGGATTAGTTGCAAAAAACTTCATTGAGGATGCCTTTTCTAATGAAAC
CAAAGATAAAGTTATTAAGTATATAAAGGATGAATTTGGTAAAATAGATT
TATTTGTTTATAGTTTAGCTGCGCCTAGGAGAAAGGACTATAAAACTGGA
AATGTTTATACTTCAAGAATAAAAACAATTTTAGGAGATTTTGAGGGACC
GACTATTGATGTTGAAAGAGACGAGATTACTTTAAAAAAGGTTAGTAGTG
CTAGCATTGAAGAAATTGAAGAAACTAGAAAGGTAATGGGTGGAGAGGAT
TGGCAAGAGTGGTGTGAAGAGCTGCTTTATGAAGATTGTTTTTCGGATAA
AGCAACTACCATAGCATACTCGTATATAGGATCCCCAAGAACCTACAAGA
TATATAGAGAAGGTACTATAGGAATAGCTAAAAAGGATCTTGAAGATAAG
GCTAAGCTTATAAATGAAAAACTTAACAGAGTTATAGGTGGTAGAGCCTT
TGTGTCTGTGAATAAAGCATTAGTTACAAAAGCAAGTGCATATATTCCAA
CTTTTCCTCTTTATGCAGCTATTTTATATAAGGTCATGAAAGAAAAAAAT
ATTCATGAAAATTGTATTATGCAAATTGAGAGAATGTTTTCTGAAAAAAT
ATATTCAAATGAAAAAATACAATTTGATGACAAGGGAAGATTAAGGATGG
ACGATTTAGAGCTTAGAAAAGACGTTCAAGACGAAGTTGATAGAATATGG
AGTAATATTACTCCTGAAAATTTTAAGGAATTATCTGATTATAAGGGATA
CAAAAAAGAATTCATGAACTTAAACGGTTTTGATCTAGATGGGGTTGATT
ATAGTAAAGACCTGGATATAGAATTATTAAGAAAATTAGAACCTTAA

Example amino acid sequence:

MIVKAKFVKGFIRDVHPYGCRREVLNQIDYCKKAIGFRGPKKVLIVGASS
GFGLATRISVAFGGPEAHTIGVSYETGATDRRIGTAGWYNNIFFKEFAKK
KGLVAKNFIEDAFSNETKDKVIKYIKDEFGKIDLFVYSLAAPRRKDYKTG
NVYTSRIKTILGDFEGPTIDVERDEITLKKVSSASIEEIEETRKVMGGED
WQEWCEELLYEDCFSDKATTIAYSYIGSPRTYKIYREGTIGIAKKDLEDK
AKLINEKLNRVIGGRAFVSVNKALVTKASAYIPTFPLYAAILYKVMKEKN
IHENCIMQIERMFSEKIYSNEKIQFDDKGRLRMDDLELRKDVQDEVDRIW
SNITPENFKELSDYKGYKKEFMNLNGFDLDGVDYSKDLDIELLRKLEP

The nucleotide sequence contained in the nucleic acid of the present invention may include a nucleotide sequence having an identity of at least 70% with one or more of the exemplary ALDH, ADH, ADHE, AOR, PTA, ACK, COAT-A, COAT-B, ADC, SADH, BCD, and TER nucleotide sequences set forth above and having one or more of the respective activities described above (e.g., an activity of catalyzing the reduction of crotonyl-CoA into butyryl-CoA by reducing the carbon-carbon double bond in crotonyl-CoA). Preferably, for example, the nucleic acid comprises a nucleotide sequence having an identity of at least 75%, more preferably 80% or more (e.g., 85% or more, more preferably 90% or more, and most preferably 95%, 98%, or 99% or more) with one or more of the exemplary ALDH, ADH, ADHE, AOR, PTA, ACK, COAT-A, COAT-B, ADC, SADH, BCD, and TER nucleotide sequences set forth above. The nucleotide sequences of the invention may have one or more nucleotide deletions, substitutions, or insertions relative to an exemplary nucleic acid sequence of the invention. For example, 1-300, 1-200, 1-100, 2-90, 3-80, 4-70, 5-50, 40, 30, 20, 10, 9, 8, 7, or 6 modifications may be made relative to one or more of the above ALDH, ADH, ADHE, AOR, PTA, ACK, COAT-A, COAT-B, ADC, SADH, BCD, and TER nucleotide sequences.

Similarly, the protein encoded by a nucleic acid of the present invention may be any protein having an identity of at least 70% with one or more of the exemplary ALDH, ADH, ADHE, AOR, PTA, ACK, COAT-A, COAT-B, ADC, SADH, BCD, and TER amino acid sequences set forth above, and having one or more of the respective activities described above. Specific examples of an amino acid sequence of the protein encoded by the nucleic acid of the present invention include an amino acid sequence having an identity of 75% or more, preferably 80% or more, more preferably 85% or more, and most preferably 90% or more (e.g., 95% or more, furthermore 98% or more) with the exemplary ALDH, ADH, ADHE, AOR, PTA, ACK, COAT-A, COAT-B, ADC, SADH, BCD, or TER amino acid sequence set forth above. The polypeptide sequences of the invention may have one or more amino acid deletions, substitutions, or insertions relative to an exemplary amino acid sequence of the invention. For example, 1-100, 1-90, 2-80, 3-70, 4-60, 5-50, 40, 30, 20, 10, 9, 8, 7, or 6 amino acid modifications may be made relative to an exemplary ALDH, ADH, ADHE, AOR, PTA, ACK, COAT-A, COAT-B, ADC, SADH, BCD, and TER amino acid sequences insofar as the encoded protein retains ALDH-, ADH-, ADHE-, AOR-, PTA-, ACK-, COAT-A-, COAT-B-, ADC-, SADH-, BCD-, and/or TER-activity.

### III. CULTURE AND FERMENTATION CONDITIONS

Culture and/or fermentation conditions for growth of microorganisms as described herein or for use in methods as set forth herein are not particularly limited, and may be selected as appropriate depending on the microorganism to be cultured as well as the bioproduct or bioproducts to be generated. For example, strains may be grown in clostridial growth medium (CGM).

In an embodiment, CGM consists of the following:

KH₂PO₄: 0.75 g/l

K₂HPO₄.3H₂O: 0.98 g/l

NaCl: 1.0 g/l

MgSO₄: 0.35 g/l

MnSO₄H₂O: 0.01 g/l

FeSO₄.7H₂O: 0.01 g/l

4-Aminobenzoic acid: 0.004 g/l

Asparagine: 2.0 g/l

Yeast extract: 5.0 g/l

(NH₄)₂SO₄: 2.0 g/l

Sodium acetate: 2.46 g/l; and

Glucose: 80.0 g/l.

Certain strains may be grown under aerobic or anaerobic conditions, as would be known to those of skill in the art. Other strains may require anaerobic growth conditions. Gas mixtures for anaerobic growth conditions may comprise, for example, 10% CO₂-5% H₂-85% N₂, or 80% H₂ 20% CO₂, or 80% N₂-20% CO₂, or 80% N₂-10% CO₂-10% H₂.

### IV. EXAMPLES

All strains were cultivated in an anaerobic chamber with an atmosphere of 10% CO₂, 5% H₂, and the balance of N₂ at 37° C. Individual colonies were selected from a solid agar plate and placed in the indicated liquid medium with appropriate antibiotics: 5 μg/ml thiamphenicol for deletion strains and 5 pg/ml clarithromycin for plasmid-harboring strains. Solid agar plates for C. acetobutylicum were 2xYTG (pH 5.8) with 15 g/l of agar. The medium 2xYTG consists of:

NaCl: 10 g/l

Tryptone: 10 g/l

Yeast extract:

### EXAMPLE 1

### Crotyl Alchohol Production in C. acetobutylicum

C. acetobutylicum was genetically engineered to produce more crotyl alcohol. The bcd gene (CA_C2711) was deleted from the chromosome to generate the strain Abcd. In addition, a plasmid, called pTHCA, over expressing the genes thl (CA_C2783), hbd (CA_C2708), crt (CA_C2712), and adhE1 (CA_P0162), was introduced into the Abcd strain.

A total of three strains were tested: C. acetobutylicum ATCC 824 [WT], C. acetobutylicum Δbcd [ΔBCD], and C. acetobutylicum Δbcd (pTHCA) [ΔBCD (pTHCA)]. Each strain was grown in 10 ml of a clostridial growth medium (CGM) anaerobically at 37° C. Endpoint samples were taken after 5 days of growth. Metabolite concentrations are presented in Table 1.

TABLE 1
End point metabolite concentrations of crotyl alcohol
producing strains of C. acetobutylicum.

| | Concentration of crotyl alcohol |
|---|---|
| Strain | (mg/l) |
| WT | 20.3 |
| ΔBCD | 41.1 |
| ΔBCD (pTHCA) | 78.6 |

As can be seen from Table 1, the concentration of crotyl alcohol was increased in the C. acetobutylicum strain in which the bcd gene was deleted. The highest concentration of crotyl alcohol was obtained with the C. acetobutylicum strain in which the bcd gene was deleted and in which the thl, hbd, crt, and adhE2 genes were overexpressed.

1. A non-naturally occurring microbial organism capable of converting acetyl-CoA into crotyl alcohol, wherein at least one of the following genes are deleted, disrupted or silenced and/or expression from at least one of the following genes is disrupted or silenced:
i. Butyryl-CoA dehydrogenase (BCD); and/or
ii. Trans-2-enoyl-CoA reductase (TER).

2, A microbial organism according to claim 1, comprising a disrupted, deleted, or mutated BCD and/or TER gene.

3. A microbial organism according to claim 1, wherein disruption or silencing of expression includes disruption or silencing of RNA transcription and/or protein translation.

4. A microbial organism according to claim 1, wherein disruption or silencing of expression comprises protein translation silencing using RNA interference.

5. A microbial organism according to claim 1, comprising at least one exogenous nucleic acid encoding one or more of the following enzymes fbr producing crotyl alcohol from crotonyl-CoA:
A. Acetaldehyde dehydrogenase;
B. Alcohol dehydrogenase;
C, Bifunctional acetaldehyde/alcohol dehydrogenase;
D. Aldehyde oxidoreductase;
E. Pbosphotransacetylase; and/or
F. Acetate kinase.

6. A microbial organism according to claim 5 for further producing acetone and/or isopronanol, comprising at least a second exogenous nucleic acid encoding one or more acetone pathway enzymes and/or one or more isopropanol pathway enzvmes, comprising
G. CoA-transferase subunit A;
H. CoA-trans (erase subunit B;
I. Acctoacetate decarboxylase; and/or
J. Secondary alcohol dehydrogenase.

7.-9. (canceled)

10. A microbial organism according to claim 6, comprising exogenous nucleic acids encoding each of the enzymes
(i) A, B, G, H, and I;
(ii) C, G, H, and I;
(iii) B, D, E, F, G, H, and I;
(iv) C, D, E, F, G, H, and I;
(v) A, B, C, G, H, and I;
(vi) A, B, C, D, E, F, G, H, and I;
(vii) A, B, C, D, E, F, G, H, and I.

11.-16. (canceled)

17. A microbial organism according to claim 5, comprising exogenous nucleic acids encoding each of the enzymes
(i) A, B, G, H, I, and J;
(ii) C, G, H, I, and J;
(iii) B, D, E, F, G, H, I, and J;
(iv) C, D, E, F, G, H, I, and J;
(v) A, B, C, G, H, I, and J;
(vi) A, B, D, E, F, G, H, I, and J; or
(vii) A, B, C, D, E, F, G, H, I, and J.

18.-23. (canceled)

24. A microbial organism according to claim 1, comprising two, three, four, five, six, seven, eight, nine, or ten exogenous nucleic acids.

25. (canceled)

26. A microbial organism according to claim 1, wherein said organism is an acetogenic bacterium.

27. A method of producing crotyl alcohol, acetone and/or isopropanol comprising culturing a microbial organism according to claim 1 on a growth substrate, optionally comprising a carbohydrate, under conditions to form a broth comprising crotyl alcohol, acetone and/or isopropanpl.

28. A method of producing crotyl alcohol and acetone, comprising culturing a microbial organism according to claim 6 on a growth substrate, under conditions to form a broth comprising crotyl alcohol and acetone, wherein the acetone to crotyl alcohol molar ratio in said broth is in the range from 0.1 to 0.95.

29. (canceled)

30. A method of producing crotyl alcohol and isopropanol, comprising culturing a microbial organism according to claim 17 on a growth substrate, under conditions to form a broth comprising crotyl alcohol and isopropanol, wherein the isopropanol to crotyl alcohol molar ratio in said broth is in the range from 0.1 to 0.95.

31.-35. (canceled)

36. A method according to claim 27, wherein said growth substrate comprises at least one gaseous compound, optionally exogenously added.

37. (canceled)

38. A method according to claim 36, wherein said at least one gaseous compound is selected from a group consisting of CO, CO2, H2 and combinations thereof.

39. A method according to claim 27, wherein said growth substrate comprises a carbohydrate in combination with at least one of a one-carbon molecule and/or a gaseous compound.

40.-41. (canceled)

42. A method according to claim 27, comprising supplementing pressurized CO2, pressurized CO, pressurized H2, or a combination thereof to said growth substrate.

43. A method according to claim 42, wherein said culturing is conducted at a pressure in the range between 1 atm and 5 atm.

44.-45. (canceled)

46. A method according to claim 27, comprising at least partially separating crotyl alcohol from said broth to form separated crotyl alcohol, at least partially separating acetone frortl said broth to form separated acetone and/or at least partially separating isopropanol from said broth to form separated isopropanol.

47.-48. (canceled)

49. A method according to claim 46, wherein said separating comprises liquid-liquid extraction.

50. A method according to claim 46, further comprising dehydrating said separated crotyl alcohol to form butadiene.

51.-105. (canceled)
